(19) United States
(12) Patent Application Publication
(10) Pub. No.: US 2010/0286049 A1
(43) Pub. Date: Nov. 11, 2010

(54) POLYCYSTIC KIDNEY DISEASE-RELATED GENE AND USE THEREOF

(75) Inventors: Yuko Wakamatsu, Nagoya-shi (JP); Hisashi Hashimoto, Nagoya-shi (JP)

Correspondence Address: OLIFF & BERRIDGE, PLC, P.O. BOX 320850, ALEXANDRIA, VA, 22320-4850, US

(73) Assignee: NATIONAL UNIVERSITY CORPORATION NAGOYA UNIVERSITY, NAGOYA-SHI (JP)

(21) Appl. No.: 12/227,528

(22) PCT Filed: May 31, 2006
(86) PCT No.: PCT/JP2006/310862

§ 371 Date: Nov. 12, 2009

(51) Int. Cl. A61K 38/17; C12Q 1/68; G01N 33/53; C12N 5/10; C12N 15/63; A61K 31/7088; C07K 14/425; C07K 16/18; C07H 21/04; A61P 13/12

(52) U.S. Cl. 514/15.4; 435/6; 435/7.1; 435/325; 435/320.1; 514/44 R; 514/44 A; 530/350; 530/387.9; 536/23.5; 536/24.5

(57) ABSTRACT

A major gene of a pc mutant of a Japanese Medaka fish (Oryzias latipes) has been identified. Based on this finding, use of the gene is provided. A gene responsible for polycystic kidney in a Japanese Medaka fish is identified in the chromosomal region of the fish by performing a positional cloning on a pc mutant of the fish. The pc gene is a transcription factor having five C2H2-type zind finger regions, and is considered to be a gene homologous to human Gli-similar 3 (Glis3) gene since it shows a high homology in the zinc finger regions.

### TECHNICAL FIELD

The present invention relates to a protein having the activity of regulating the onset and progression, etc., of polycystic kidney disease, a nucleic acid molecule encoding the protein, and the uses thereof.

### BACKGROUND ART

Many of the main organs in the bodies of animals comprise “tubes” as the basic unit thereof. Kidney is one such typical organ. However, the molecular mechanism underlying “tube” formation is barely understood.

Subtypes of polycystic kidney disease (PKD) in humans include autosomal recessive polycystic kidney disease (ARPKD) and autosomal dominant polycystic kidney disease (ARPKD). Among these subtypes, ADPKD is a widespread genetic disease, and it is estimated that 100,000 to 200,000 persons are afflicted with this disease in Japan. ADPKD is a systemic disorder that not only causes cysts in the kidneys, but also in the liver, pancreas, and spleen. The medical histories of ADPKD patients frequently include cerebral hemorrhage, etc., and hypertension is a complication thereof in more than half the cases. The formation of kidney cysts, which is the most striking feature of ADPKD, causes renal hypertrophy and diminished urine concentration capability. These symptoms progress with aging in ADPKD patients, and because no effective mode of therapy exists, ultimately the kidneys fail, necessitating dialysis and kidney transplantation. The pathology of cyst formation in ADPKD is manifested by hypertrophy of the renal tubes, i.e., an abnormality in the regulation and maintenance of tube diameters.

The PKD1 gene at location 16p13.3 on human chromosome 16 and the PKD2 gene at location 4q21-23 of human chromosome 4 have been identified as responsible genes of ADPKD, and these encode the proteins polycystin-1 (PC1) and polycystin-2 (PC2) respectively (Wilson P D, et al., N Engl J Med, 8 Jan. 2004, 350(2): 151-64). In addition, the responsible gene(s) have been cloned in a murine polycystic kidney disease model (Liang J D, et al., J Formos Med Assoc. June 2003, 102(6):367-74). It has been learned that these proteins are present in the cilia located at the tips of tubular epithelial cells, and it is predicted that tubule hypertrophy due to ciliary dysfunction is one of the cause of the onset of ADPKD (Boletta A, et al., Trends Cell Biol. September 2003; 13(9): 484-92, Review). However, the detailed mechanism of the onset of ADPKD is still unknown.

### DISCLOSURE OF THE INVENTION

The inventors have discovered that there is a mutant medaka fish (Oryzias latipedes) that expresses a phenotype strongly resembling the pathology of human ADPKD (hereinafter, the medaka mutant is referred to as the medaka pc mutant). In this medaka pc mutant, hypertrophy of renal tubule diameter begins soon after hatching; the renal hypertrophy progresses together with aging, whereas the kidney weight in adults becomes at least 100-fold greater than in normal medaka, and death occurs a few months after hatching. The kidneys of this mutant are enormous and contain complex, branching cysts. The cells constituting the cysts exhibit the histological feature of squamous metaplasia, and their histology is remarkably similar to that of human ADPKD cells.

In general, the structure and function of organs in medaka and other small fish have a high degree of commonality with those in humans. With respect to the kidney, the medaka kidney has a plurality of nephron units comprising a glomerulus and proximal and distal tubules just as in humans. In addition, the genomic controls involved in kidney development in mammals are conserved in medaka. Moreover, with respect to the genome, not only are many genes in both humans and fish analogous, but synteny, i.e., the co-localization of a plurality of homologous genes in the same linkage group or on the same chromosome, also exists over a wide range of chromosomes. Furthermore, medaka have many merits in terms of research: their eggs are transparent, they lay eggs throughout the year in an artificial environment, the time between generations is relatively short; i.e. 2 to 3 months, they are easy to raise, and their maintenance costs are extremely low. Based on the above conditions, medaka can serve as a tube diameter regulation model in developmental biology, and also as a human disease model. More specifically, the medaka pc mutant is mesonephric and the mesonephros persists into adulthood, so it is expected to serve as the only good disease model for PKD among small fish.

However, in the course of their research the inventors learned that the renal tubule epithelial cells of the medaka pc mutant are ciliated, and no abnormalities were found in the expression of mRNA molecules encoding polycystin-1 and polycystin-2. They also discovered that a mutation in a new gene heretofore undiscovered in the medaka pc mutant may be the cause of the disease. It is expected that the identification of the responsible gene in the medaka pc mutant will contribute not only to understanding the “tube” forming control mechanism in animals, but also to understanding the mechanism of the onset of PKD in humans. Even more so, it is expected that identification of the responsible gene will contribute to the diagnosis and treatment of PKD in humans and other animals.

Thus, an object of the present invention is to identify the causative gene in the medaka pc mutant, and a further object of the present invention is not only to understand the mechanism of onset of PKD therein, etc., but also to search for a drug to treat PKD in animals and use such a drug in an agent for the diagnosis and treatment of PKD.

The inventors used positional cloning techniques in the medaka pc mutant to identify and isolate the gene responsible for polycystic kidney disease in medaka from its chromosomal region. In other words, the inventors identified the linkage group of the putative responsible gene (hereinafter, referred to as the pc gene) in the medaka pc mutant, and then prepared a high resolution chromosome map of the vicinity of the pc gene, began chromosome walking from the nearest marker, and identified a BAC clone straddling the pc gene locus. Next, shotgun cloning of that clone was performed and a comparison was made with the genome of the fugu puffer fish (Takifugu rubripes), and the potential pc gene locus was narrowed to two regions. Then, an expression analysis of these regions was performed in wild type and pc mutant medaka. Based on the evidence that an insertion or deletion mutation is present on the 3′ end of one of the two aforementioned genes in the mutant and that the mRNA transcription product of that gene is not detected, etc., the gene causing that mutation was identified as the medaka pc gene. The pc gene is thought to be a transcription factor having five C2H2 zinc finger motifs, and based on the high level of homology in the zinc finger motifs, it is thought to be a homologue of the human Gli-similar3 (Glis3) gene. The present invention provides the following means.
(1). DNA selected from:
   (a) DNA encoding a protein having the amino acid sequence of SEQ ID NO: 2 or 4;
   (b) DNA encoding a protein having an amino acid sequence in which one or a plurality of amino acid residues is substituted into, deleted from, and/or added to the amino acid sequence of SEQ ID NO: 2 or 4, and having substantially the same activity thereof;
   (c) a nucleic acid molecule having the base sequence of SEQ ID NO: 1 or 3; and
   (d) DNA that hybridizes under stringent conditions with DNA having a base sequence complementary to the base sequence of SEQ ID NO: 1 or 3.
(2) DNA encoding a part of the protein described in (a) above or the protein described in (b) above.
(3) DNA that hybridizes with the nucleic acid molecule according to (1) or (2), or a complementary strand thereof.
(4) DNA having a sequence identical to a continuous base sequence of 100 or fewer bases of the DNA according to (1) or (2), or a complementary strand thereof
(5) A detection agent containing the DNA according to any of (1) to (4).
(6) Antisense DNA to the DNA according to (1) or (2).
(7) A vector containing the DNA according to (1) or (2).
(8) The vector according to (7), containing RNA equivalent to the DNA sense strand as the DNA.
(9) A transformant having the vector according to (7) or (8) inserted therein.
(10) A transformant in which expression of the endogenous DNA according to any of (1) to (4) is inhibited.
(11) A polypeptide that is either a protein or part thereof selected from a group consisting of:
   (a) a protein having the amino acid sequence of SEQ ID NO: 2 or 4; and
   (b) a protein having an amino acid sequence in which one or a plurality of amino acid residues is substituted into, deleted from, and/or added to the amino acid sequence of SEQ ID NO: 2 or 4, and having substantially the same activity thereof.
(12) An antibody to the polypeptide according to (11).
(13) A detection agent containing the antibody according to (12).
(14) A drug for prevention or treatment of polycystic kidney disease containing DNA encoding a GLIS3 protein or a part thereof.
(15) The drug according to (14), wherein the DNA encoding the GLIS3 protein is DNA selected from the following group:
   (a) DNA encoding a protein having any of the amino acid sequences selected from the amino acid sequences of SEQ ID NOs: 2, 4, 6, and 8;
   (b) DNA encoding a protein having an amino acid sequence in which one or a plurality of amino acid residues is substituted into, deleted from, and/or added to any of the amino acid sequences selected from the amino acid sequences of SEQ ID NOs: 2, 4, 6, and 8, and having substantially the same activity thereof;
   (c) DNA having any of the base sequences selected the base sequences of SEQ ID NOs: 1, 3, 5, and 7; and
   (d) DNA that hybridizes under stringent conditions with a base sequence complementary to any of the base sequences selected the base sequences of SEQ ID NOs: 1, 3, 5, and 7.
(16) A method for prevention or treatment of polycystic kidney disease comprising a step of administering an effective amount of DNA encoding a GLIS3 protein or part thereof to an animal.
(17) A diagnostic agent for polycystic kidney disease containing DNA encoding a GLIS3 protein or a part thereof, or antisense DNA having a base sequence that is complementary to or substantially complementary to the base sequences of those DNA molecules.
(18) A method for diagnosis of polycystic kidney disease containing DNA encoding a GLIS3 protein or a part thereof, or antisense DNA having a base sequence that is complementary to or substantially complementary to the base sequences of those DNA molecules.
(19) A screening method for a drug for prevention or treatment of polycystic kidney disease that utilizes DNA encoding a GLIS3 protein or a part thereof, or antisense DNA having a base sequence that is complementary to or substantially complementary to the base sequences of those DNA molecules.
(20) A screening method for a drug for prevention or treatment of polycystic kidney disease using cells that express a Glis3 gene.
(21) A screening method for a drug for prevention or treatment of polycystic kidney disease using cells in which expression of a Glis3 gene is inhibited.
(22) The screening method for a drug for prevention or treatment of polycystic kidney disease according to (21), using a transformant animal in which expression of a Glis3 gene is inhibited.
(23) A drug for prevention or treatment of polycystic kidney disease containing a GLIS3 protein, a part thereof, or a salt thereof.
(24) The drug according to (23), wherein the GLIS3 protein is:
   (a) a protein having any of the amino acid sequences selected from the amino acid sequences of SEQ ID NOs: 2, 4, 6, and 8; or
   (b) a protein having an amino acid sequence in which one or a plurality of amino acid residues is substituted into, deleted from, and/or added to any of the amino acid sequences selected from the amino acid sequences of SEQ ID NOs: 2, 4, 6, and 8 and having substantially the same activity thereof.
(25) A method for prevention or treatment of polycystic kidney disease comprising a step of administering an effective amount of a GLIS3 protein, a part thereof, or a salt thereof to an animal.
(26) A screening method for a drug for prevention or treatment of polycystic kidney disease using a GLIS3 protein, a part thereof, or a salt thereof.
(27) A screening method for a drug for prevention or treatment of polycystic kidney disease, using cells capable of expressing a GLIS3 protein or a part thereof.
(28) A diagnostic agent for polycystic kidney disease containing an antibody to a GLIS3 protein, a part thereof, or a salt thereof.
29. A diagnostic method for polycystic kidney disease utilizing an antibody to a GLIS3 protein, a part thereof, or a salt thereof.

### BRIEF DESCRIPTION OF THE DRAWINGS

FIG. 1 shows the genetic map of the candidate region of the pc mutation. In the figure, (a) represents a recombination map of linkage group 12, and (b) represents the start of chromosome walking using BAC with polymorphic marker AU171175 as the origin. The pc gene locus was approached in the sequence of BAC clones 184A3, 198E6, 201K4, and 174E15. The terminal sequence of each BAC insert was mapped, and these were named 184A3F, 231H8R, 201K4F, and 174E15R, respectively. The number of recombination between the pc gene locus and sequences 201K4F and 174E15R was 1 and 3, respectively, and it was confirmed that the pc gene locus is present on BAC clone BAC174E15. (c) shows the genes on BAC174E15 revealed by shotgun sequencing. From the left, the gene regions are pc, RFX3, SMAD4, BMP10, and catenin-ARVCF. (d) is the intron/exon structure of the wild type pc gene. The structure shows that the gene comprises at least 10 exons. It can be seen that exon 3 is spiced selectively. (d) shows that in the pc gene region of the pc mutant, exons 5-10 behind exon 4 are missing.

FIG. 2 shows the expression analysis of the mRNA of pc and c78 (RFX3). The mRNA was detected in medaka one month after hatching. A c80-67 primer set was used for pc and a c78 primer set was used for RFX3. In the pc mutant, pc mRNA was not detected.

FIG. 3 shows an expression map of pc mRNA. The pc mRNA was detected in the kidneys of the pc mutant and the OR strain (wild type) by northern blotting. A band seen in the OR strain was not found in the pc mutant.

FIG. 4 shows cDNA of the pc gene. The underlined portion is the selective splicing site. Two types of mRNA with different start codons are produced by selective splicing. The methionine due to each start codon is shown by an underline. In the pc mRNA of the pc mutant, the portion of exons 5-10 is changed to a “base sequence continuing behind exon 4 in the mutated pc mRNA of the pc mutant.” The bases at the 3′ and 5′ ends of each exon are enclosed in rectangles, and a slash (/) is inserted to show the exon boundaries.

FIG. 5 shows time-course changes in pc mRNA in a medaka embryo. The upper band is pc and the lower band is a control (EF-1α).

FIG. 6 shows the organ distribution of pc mRNA in adult medaka. Total RNA prepared from kidney, liver, gut, gill, ovary, and spleen was investigated using RT-PCR.

FIG. 7 shows whole-mount in situ hybridization of pc mRNA. The photo shows the ventral view of a medaka at day 5 after hatching. The internal organs such as the gut, etc., have already been removed.

FIG. 8 shows a comparison of pc mRNA in the kidneys of the pc mutant and the OR strain. A comparison of the presence or absence of the regions amplified by the primer sets shown in the figure was performed by RT-PCR on total RNA prepared from the kidneys of the pc mutant and the OR strain (wild type).

FIG. 9 shows a comparison of the pc gene regions of the pc mutant and the OR strain. A comparison showing whether or not each region is amplified by the primer sets shown in the figure was made carrying out PCR on genomic DNA prepared from the pc mutant and the OR strain.

FIG. 10 shows a comparison of the amino acid sequences of the medaka pc and human Glis3 proteins. Matching and analogous amino acid residues are indicated by hatched areas.

FIG. 11 is a comparison of the zinc finger domains of the pc protein and other similar proteins. The C2H2 zinc finger domains of medaka S935, medaka S2012, human Glis1, human Glis3, medaka pc, human Gli2, human Glis2, and human Zic1 are extracted and compared.

FIG. 12 shows the level of homology of the zinc finger motifs and a phylogenetic tree of the pc protein and other analogous proteins.

FIG. 13 shows the synteny of the region surrounding the medaka pc gene with regions on human chromosome 9 and mouse chromosome 19.

FIG. 14 describes the mRNA probe sites using whole-mount in situ hybridization. The top row is wild type pc mRNA and the bottom row is pc mRNA from the pc mutant.

FIG. 15 shows the results of investigating the expression of pc mRNA in the wild type medaka kidney using whole-mount in situ hybridization. The top row shows ventral photographs of the medaka kidney region at 0, 5, 20, and 30 days after hatching. The bottom row shows photographs of anterior cross-sections at day 5 after hatching. Arrows 1 and 2 in the top row correspond to sections 1 and 2 on the bottom row.

FIG. 16 shows the results of investigating the expression of pc mRNA in the pc mutant medaka kidney. The top row shows ventral photographs of the medaka kidney region at 0, 5, and 10 days after hatching. The bottom row shows anterior photographs of anterior cross-sections at day 5 after hatching. The left shows a strong phenotype individual and the right shows a weak phenotype individual. Arrows 1 and 2 in the top row correspond to sections 1 and 2 on the bottom row.

FIG. 17 shows the phenotype statistics when the pc gene is knocked down with an antisense oligonucleotide. Twenty-five individuals were randomly selected from hatched individuals, and the renal histology thereof was examined.

FIG. 18 shows examples when the phenotype manifested by pc knockdown is classified by site of cyst formation. The arrows show glomerular cysts, and the asterisks (*) show tubular cysts.

FIG. 19 shows the phenotype statistics for a pc and S2012 (glis1) double knockdown. Even under the dissecting microscope before sectioning, cysts could be seen in all 8 individuals with this phenotype.

FIG. 20 shows one example of the phenotype manifested by the pc and S2012 (glis1) double knockdown when classified by site of cyst formation. In the top row the arrows show glomerular cysts, and the asterisks (*) show tubular cysts. In the bottom row the arrows show cysts that can be recognized even under a dissecting microscope.

FIG. 21 shows the organ distribution of S2012 mRNA in adult medaka. Total RNA prepared from kidney, liver, gut, gill, ovary, and spleen was investigated using RT-PCR.

FIG. 22 shows the pc mutation caused by insertion of a transposon.

The transposon is inserted at the 5264th base from the 5′ side of intron 4 (5727 bases long). The arrow shows the repeat sequences at the ends of the transposon.

### BEST MODE FOR CARRYING OUT THE INVENTION

The present invention is described more in detail below.

The present invention provides polynucleotide encoding a polypeptide involved in polycystic kidney disease (PKD) and causing the onset or progression thereof, the polypeptide itself, and the uses thereof. As noted above, the inventors have identified the medaka pc gene, i.e. the responsible gene of PKD in medaka, that was isolated using positional cloning.

In the medaka pc mutant used in the present invention, a deletion or insertion has occurred due to transposition at the 3′ end of the pc gene that had been isolated by the inventors, and therefore the original polypeptide thereof is not expressed in the pc mutant. In a normal medaka, the pc gene is strongly expressed in the kidneys of adults. In other words, it has been confirmed that the pc gene has the activity of regulating the onset and/or progression of PKD. Therefore, it is possible to use the pc gene to search for novel drugs that can be used to elucidate the organ-forming mechanism, and the onset and progression of PKD, and to search for new drugs to treat PKD.

The cDNA base sequences of the pc gene are shown in SEQ ID NOs: 1 and 3, and the amino acid sequences of the polypeptides encoded thereby are shown in SEQ ID NOs: 2 and 4, respectively. These two species of polypeptides originate due to selective splicing from the same genome of the medaka pc gene.

The amino acid sequences of the pc proteins represented by SEQ ID NO: 2 and SEQ ID NO: 4 are both zinc finger type transcription factors having five C2H2 zinc finger motifs. These proteins are 48% and 49% homologous, respectively, with the human Gli-similar3 (GLIS3) protein, and are 50% and 52%, homologous, respectively, with the mouse GLIS3 protein. The homology between animal species is particularly high in the zinc finger motifs, and the homology between the pc protein (both pc-a and pc-b) and the human GLIS3 protein is 87.4% in these regions. This is significantly higher homology than the homology with other related zinc finger family members Gli and Zic, and this finding supports the belief that the medaka pc gene is a homologue of the human Glis3 gene. In addition, the human Glis3 gene is neither identical to nor homologous with the PKD 1 gene or PKD2 gene, which have been identified as the causative genes of PKD. Therefore, the Glis3 gene is a novel causative gene of PKD. In this description, the term “Glis3 gene” refers to a gene encoding the GLIS3 protein.

### (Polynucleotide)

The polynucleotide of the present invention is defined as a polynucleotide having the base sequence of SEQ ID NO: 1 or SEQ ID NO: 3, and as a polynucleotide encoding a polypeptide having the amino acid sequence of SEQ ID NO: 2 or SEQ ID NO: 4.

The polynucleotide of the present invention includes a polynucleotide encoding a homologous gene in a human or other animal that has a high level of homology with the amino acid sequence of SEQ ID NO: 2 or SEQ ID NO: 4. The polypeptide encoded by the base sequence of SEQ ID NO: 1 or 3 of the present invention is a zinc finger type transcription factor, and based on the high level of homology in the zinc finger regions thereof, is a homologue of the human Gli-similar3 (Glis3) gene. It is also a homologue with the mouse Glis3 gene. SEQ ID NO: 6 shows the amino acid sequence of the human GLIS3 protein, and SEQ ID NO: 7 shows the amino acid sequence of the mouse GLIS3 protein. Therefore, the polynucleotide of the present invention includes a polynucleotide having the base sequence of SEQ ID NO: 5 or 7 and a polynucleotide encoding a protein having the amino acid sequence of SEQ ID NO: 6 or 8. Utilization of the human and mouse Glis3 gene is effective for the detection, diagnosis, screening for drugs, and treatment related to PKD in humans, mice, and other mammals.

The polynucleotide of the present invention also includes a polynucleotide encoding a protein having an amino acid sequence identical to an amino acid sequence of SEQ ID NOs: 2, 4, 6, and 8, or an amino acid sequence substantially identical thereto.

The polynucleotide of the present invention also includes a polynucleotide encoding a protein wherein 1 or a plurality of amino acid residues is substituted into, deleted from, and/or added to an amino acid sequence of SEQ ID NO: 2, 4, 6, or 8 as a protein having an amino acid sequence substantially identical thereto, and having substantially the same activity as a protein having the original amino acid sequence thereof The term “substantially the same activity” includes PKD regulatory activity, etc. The extent of the activity thereof is not particularly limited herein. It is possible that such a polynucleotide will be discovered as naturally occurring in a mutant, in other medaka species and small fish, and in other animal species.

A nucleic acid molecule encoding a protein with a modified amino acid sequence can be artificially prepared, and the methods therefor are well known to persons skilled in the art. It is possible, for example, to use a commercially available kit therefor. For example, such preparation can be carried out using a “Transformer Site-directed Mutagenesis Kit” and “ExSite PCR-Based Site-directed Mutagenesis Kit” (Clontech Laboratories) for mutation or substitution, and a “Quantum Leap Nested Deletion Kit” (Clontech Laboratories) for deletion. The number of amino acid residues deleted, substituted or added by a well-known method such as site-directed mutagenesis and the like can range from 1 to several dozen; preferably 1 to 20, more preferably 1 to 10, and even more preferably 1 to 5. Preferably, the modification of amino acids will involve conservative substitution. The DNA of the present invention also includes degenerative mutants.

Amino acid sequences that are substantially identical to a protein having an amino acid sequence of SEQ ID NO: 2, 4, 6, or 8 include, for example, an amino acid sequence having 50% or more homology with any of the amino acid sequences of SEQ ID NOs: 2, 4, 6, and 8, more preferably 60% or more homology, even more preferably 70% or more homology, and even more preferably 80% or more homology; furthermore, a sequence with 90% or more homology is even more preferred, and one with 95% or more homology is most preferred. The level of homology in the zinc finger region is preferably 70% or more, more preferably 75% or more, even more preferably 80% or more, and most preferably 90% or more.

The polynucleotide of the present invention also includes a polynucleotide capable of hybridizing under stringent conditions with DNA or another polynucleotide complementary to a base sequence of SEQ ID NO: 1, 3, 5, or 7. The term “polynucleotide capable of hybridizing under stringent conditions” refers to a nucleic acid molecule obtained using colony hybridization, plaque hybridization, or Southern blot hybridization, etc., using DNA, etc., comprising the base sequence of SEQ ID NO: 1, 3, 5, or 7 or a part thereof as a probe. More specifically, this includes DNA that can be identified by performing hybridization at 42° C. in the presence of 50% formamide, 6× Denhardt solution, 5×SSC solution, and 1% SDS using a filter having DNA from a colony or plaque immobilized thereon, and then rinsing the filter with 0.2×SSC solution under the condition of 65. The hybridization can be performed by following the methods described in Molecular Cloning, A Laboratory Manual, Second Edition, Cold Spring Harbor Laboratory Press (1989) (hereinafter, abbreviated as Molecular Cloning, Second Edition), Current Protocols in Molecular Biology, John Wiley & Sons (1987-1997) (hereinafter, abbreviated as Current Protocols in Molecular Biology), or DNA Cloning 1: Core Techniques, A Practical Approach, Second Edition, Oxford University (1995), etc. The hybridizable DNA specifically includes DNA having a base sequence that is at least 60% or more homologous with a base sequence of SEQ ID NO: 1, 3, 5, or 7 when calculated using BLAST [J. Mol. Biol., 215, 403 (1990)] and FASTA [Methods in Enzymology, 183, 63-98 (1990)], etc., preferably DNA having 70% or more homology, more preferably DNA having 80% or more homology, even more preferably DNA having 90% or more homology, and most preferably DNA having 95% or more homology.

The polynucleotide of the present invention can be obtained by using polymerase chain reaction (PCR) techniques (Saiki R K, et al., Science 230: 1350, 1985 and Saiki R K, et al., Science 239: 487, 1988). Based on these techniques, the inventors were able to isolate polynucleotides encoding polypeptides having a high level of homology with the polypeptide of the present invention from other small fish and other animals by using oligonucleotides based on the base sequence of the polynucleotide of the present invention (SEQ ID NOs: 1, 3, 5, or 7) or a part thereof.

The term that a “polypeptide has PKD regulatory activity” means that the polypeptide has the activity that circumvents or inhibits the onset and progression of PKD in animals or has the activity that enables normal kidney function to be expressed. Whether or not a polypeptide has such regulatory activity can be determined by a functional complementation test based on expression of the polypeptide in the pc mutant. It can also be determined by the screening method described below.

The polynucleotide of the present invention can also be a part of the aforementioned polynucleotide. In other words, the polynucleotide of the present invention includes a polynucleotide encoding proteins having the amino acid sequences of SEQ ID NO: 2, 4, 6, or 8, and parts of proteins that are substantially identical thereto. That is because a part of the encoded polypeptide may have PKD regulatory activity, or PKD regulatory may not be necessary for use thereof in the reagent, diagnostic agent, etc., as will later be described below.

The polynucleotide of the present invention also includes a polynucleotide that can hybridize with the above polynucleotide or its complementary strand. Such a hybridizable polynucleotide can be used as a probe in hybridization technology and as a primer in PCR technology, and can be used in a variety of diagnostic, detection, screening, and other methods based on those technologies. For hybridization the polynucleotide of the present invention does not need to be entirely complementary with the base sequence of the polynucleotide of which is the target of hybridization, and as such it being substantially complementary will suffice. The term “substantially complementary” means that it must be complementary to the extent that it can specifically hybridize with at least one part of the base sequence of the polynucleotide being the target of hybridization. The polynucleotide of the present invention can be a nucleic acid molecule having a sequence identical to a base sequence consisting of 100 or fewer continuous bases of such a nucleic acid molecule or its complementary strand. Preferably, it will be 60 or fewer bases long, and more preferably 40 or fewer bases long. On the other hand, preferably it will be 5 or more bases long, more preferably 10 or more bases long, and even more preferably 15 or more bases long.

DNA, or mRNA and other species of RNA can serve as the polynucleotide of the present invention, and it can be either double-stranded or single-stranded. If it is double-stranded, it can be double-stranded DNA, double-stranded RNA or a DNA/RNA hybrid. If it is single-stranded, the polynucleotide can also be an antisense strand. DNA encoding the GLIS3 protein can be genomic DNA, cDNA, synthesized DNA, or a genomic DNA library or cDNA library. Thus, various types of DNA molecules and RNA molecules corresponding thereto such as mRNA, etc., are included herein. Preferably the polynucleotide of the present invention is DNA. When the term “DNA or RNA of the present invention” is used below, it refers to a case wherein the polynucleotide of the various modes of the present invention is either DNA or RNA.

### (Antisense Polynucleotide)

The antisense polynucleotide of the present invention is a polynucleotide that is identical to the complementary strand of the polynucleotide of the present invention or part thereof, or one that is substantially complementary to the polynucleotide of the present invention or part thereof. By the insertion thereof into a cell, the antisense nucleic acid molecule can inhibit the expression of that nucleic acid molecule. Chemical modification on such an antisense DNA molecules and antisense RNA molecule can be performed to make the breakdown thereof in the body more difficult, or to enable passage through the cell membrane. Such molecules can also be configured into a construct containing a DNA molecule that enables expression of an antisense RNA molecule in the body. The antisense nucleic acid molecule does not need to be 100% complementary to the targeted RNA, etc., however, it is preferably for it to be 90% or more complementary, and most preferably 95% or more complementary.

### (Polypeptide)

The present invention provides a polypeptide having regulatory activity toward PKD. Such a polypeptide not only includes a protein having the amino acid sequence of SEQ ID NOs: 2 or 4, but also a protein encoded by the Glis3 gene, which is a homologue of the medaka pc gene. Examples thereof include a protein having the amino acid sequence of the Glis3 protein in humans (SEQ ID NO: 6) and in mice (SEQ ID NO: 8).

The polypeptide of the present invention includes those having an amino acid sequence wherein 1 or a plurality of amino acid residues is substituted into, deleted from, and/or added to those amino acid sequences, and having substantially the same activity as the original protein. In the present invention the proteins having the amino acid sequences of SEQ ID NOs: 2, 4, 6, or 8, and the aforementioned proteins are all grouped together and referred to as the GLIS3 protein. Not only can a protein having such a modified amino acid sequence be obtained by a variety of publicly known methods, but it can also be obtained as a recombinant protein by carrying out gene recombination techniques using the DNA molecule of the present invention. Preferably the polypeptide of the present invention will have substantially the same activity as the protein having one of the original amino acid sequences. The term “substantially the same activity” refers to regulatory activity, etc., of PKD, and the determination thereof has already been described.

A protein having an amino acid sequence wherein 1 or more amino acid residues is substituted into, deleted from, and/or added to the amino acid sequence of SEQ ID NOs: 2, 4, 6, or 6 can be obtained using the site-directed mutagenesis methods described in Molecular Cloning, Second Edition; Current Protocols in Molecular Biology; Nucleic Acids Research, 10, 6487 (1982); Proc. Natl. Acad. Sci. USA, 79, 6409 (1982); Gene, 34, 315 (1985); Nucleic Acids Research, 13, 4431 (1985); Proc. Natl. Acad. Sci. USA, 82, 488 (1985); etc., by introducing a site-directed mutation into DNA encoding a polypeptide having the amino acid sequence of SEQ ID NO: 2 or 4, for example. The number of amino acid residues deleted, substituted or added is not particularly limited herein, and a number that can be deleted, substituted, or added by a well-known method such as site-directed mutagenesis and the like can range from 1 to several dozen, but preferably will be 1 to 20, more preferably 1 to 10, and even more preferably 1 to 5. In addition, with respect to the extent of these amino acid deletions, substitutions or additions, the modified amino acid sequence will have homology of at least 60% or more, preferably 80% or more, more preferably 90% or more, and even more preferably 95% or more with the original amino acid sequence. The level of homology in the zinc finger region is preferably 70% or more, more preferably 75% or more, even more preferably 80% or more, and most preferably 90% or more.

The polypeptide of the present invention may also be a partial protein thereof. The number of amino acid residues in this mode of the polypeptide is not particularly limited herein, but preferably it has an amino acid sequence that is identical to or substantially identical to the amino acid sequence of the original protein and has substantially the same activity thereof. The term “substantially identical to the amino acid sequence” means that it has an amino acid sequence of which 1 or a plurality of amino acid residues is substituted into, deleted from, and/or added to an amino acid sequence of SEQ ID NO: 2, 4, 6, or 8, and the term “substantially the same activity” means the same as defined above.

The polypeptide of the present invention can be obtained not only by producing the same from animal cells or tissues using prior art, publicly known protein purification methods, but also by culturing a transformant that has been transformed by DNA encoding the same. In addition, it can be obtained by a prior art, publicly known chemical synthesis method.

The polypeptide of the present invention includes a GLIS3 protein or a partial salt thereof. The form of the salt is not particularly limited herein, but a pharmaceutically acceptable salt is preferred. Examples of the salt include the salts of hydrochloric acid, phosphoric acid, sulfuric acid and other inorganic acids, and the salts of acetic acid, citric acid, succinic acid, methanesulfonic acid, and other organic acids.

### (Antibody)

The antibody of the present invention is an antibody that recognizes the GLIS3 protein of the present invention or a part thereof. The antibody of the present invention includes both monoclonal antibodies and polyclonal antibodies. The antibody of the present invention can be prepared by conventional methods using the protein of the present invention or a part thereof as an antigen for antibody production. The antibody of the present invention can be used not only to purify the protein of the present invention, but can also be used to detect and quantify the protein of the present invention by western blotting, ELISA, immunofluorescence techniques, and the like, and to detect the localization thereof in cells and the body. It is possible to search for the protein of the present invention and proteins similar to such by using the antibody of the present invention. The antibody can also be synthesized by a peptide synthesizer based on SEQ ID NOs: 2, 4, 6, or 8.

### (Detection Agent)

The polynucleotide and antisense polynucleotide in the various modes of the present invention noted above can be used as an agent to detect the Glis3 gene. Examples include DNA or a part thereof encoding a protein having an amino acid sequence that is identical to or substantially identical to the amino acid sequence of SEQ ID NO: 2 or 4, DNA or a part thereof having a base sequence identical to or substantially identical to the base sequence of SEQ ID NO: 1 or 3, and a complementary strand thereto or DNA having a substantially complementary base sequence thereto. Such a detection agent can hybridize specifically with the targeted nucleic acid molecule or part thereof and be used as a probe to detect or isolate the polynucleotide of the present invention, or it can be used as a primer to amplify the same. By linking the nucleic acid to a dye, fluorescent dye, radioisotope or a linking group thereto, etc., and configuring the same so that a signal change will be generated either directly or indirectly by hybridization, whereby the presence or absence of expression of that nucleic acid molecule, and the extent of the expression thereof can be visualized through hybridization with such a probe in tissues and cells. The target of detection can be the DNA of the Glis3 gene, the mRNA transcription product thereof or other type of RNA, or cDNA.

The aforementioned antibody of the present invention can also be used as a detection agent to detect the GLIS3 protein. For example, an antibody to a protein or part thereof having an amino acid sequence identical to or substantially identical to the amino acid sequence of SEQ ID NO: 2 or 4 is a preferred detection agent for those polypeptides. Such an antibody can also be used to isolate the polypeptide of the present invention. Furthermore, by linking the antibody of the present invention to a dye, fluorescent dye, radioisotope or a linking group thereto, etc., the protein of the present invention can be detected and quantified by western blotting, ELISA, immunofluorescence techniques, and the like.

### (Diagnostic Method and Diagnostic Agent for PKD)

The various modes of polynucleotide and antisense polynucleotide of the present invention can be used for the diagnosis of PKD. In accordance with this diagnostic agent, an abnormality in the Glis3 gene and the presence or absence of the expression product thereof, as well as abnormalities therein and the extent thereof, can be measured from a variety of samples such as blood, serum, urine, tissue, cells, etc., collected from the individual to be diagnosed or a nucleic acid extract thereof, Furthermore, it is thereby possible to diagnose whether the individual has PKD or not, and also the extent of the condition. For example, in cases where there is an abnormality in the Glis3 gene and the mRNA thereof, and in cases where the level of mRNA is decreased, it is possible to diagnose that the onset of PKD is possible, or that the condition of PKD has progressed. In such measurements the cDNA, mRNA, and other polynucleotides in the sample can be analyzed by plaque hybridization, colony hybridization, Southern blotting, northern blotting, RT-PCR, DNA chip or DNA microarray, etc. Furthermore, PKD can be diagnosed by a sequence comparison (including detection of restriction enzyme sites) between the human Glis3 gene or another polynucleotide of the present invention and cDNA extracted from the individual to be diagnosed.

The antibody of the present invention can be used for the diagnosis of PKD. In accordance with this diagnostic agent containing the antibody of the present invention, the presence or absence of these proteins and the content thereof can be measured from a variety of samples such as blood, serum, urine, tissue, cells, etc., collected from the individual to be diagnosed or a nucleic extract thereof, and it is possible to diagnose whether the individual has PKD or not, and the extent of the condition. ELISA, western blotting, immunofluorescence, and tissue staining can be used for such measurements. An antibody chip can also be used therefor.

### (Vector)

The vector of the present invention incorporates the DNA of the present invention or the antisense DNA of the present invention. In accordance with a vector containing the DNA of the present invention and configured to express the GLIS3 protein or part thereof, the DNA of the present invention capable of expressing the GLIS3 protein can be carried by animal cells, small fish, or an animal. In accordance with a vector configured to express the antisense DNA of the present invention, expression of the Glis3 gene can be inhibited in cells transfected therewith. The vector can contain the sense strand of the DNA of the present invention, or an equivalent RNA molecule. Such RNA is preferably used in an RNA virus vector. The configuration of the vector can be suitably selected in accordance with the insertion thereof, etc., into the type of cells or host to be transfected with the vector.

### (Transformant)

The transformant of the present invention includes a transformant carrying the exogenous DNA of the present invention. Such a transformant can be used for production of the polypeptide of the present invention and for screening for drugs for PKD.

The cells serving as the transformant are not particularly limited herein, and both microbial cells and animal cells can be used as needed. In a case where it is intended to manufacture the GLIS3 protein, there may be cases in which usage of microorganisms is effective because of their productivity.

By inserting a vector configured to express the GLIS3 protein or part thereof into a suitable host, it is possible to obtain a transformant that produces the GLIS3 protein or part thereof. Such a transformant can be used not only to produce the GLIS3 protein, but also to screen for drugs for the prevention and treatment of PKD.

In addition, a transgenic animal expressing the GLIS3 protein or a part thereof can be prepared by employing a vector configured to express the GLIS3 protein or a part thereof in a publicly known technique for preparing a transgenic animal. Such a transgenic animal will express a high level of the GLIS protein, and will be useful for elucidating the pathology of PKD and other diseases linked to the GLIS3 protein, and the action of the GLIS3 protein. Furthermore, based on the base sequence of the Glis3 gene, a knockout animal in which expression of the Glis3 gene is inhibited (or destroyed) can be prepared. Such a knockout animal will be useful as a PKD disease model animal, and screening for drugs for the prevention or treatment of PKD can be carried out using that disease model animal. Additionally, by inserting a reporter gene into the structural gene member of the Glis3 gene, a knockout animal wherein the reporter gene is expressed by the Glis3 promoter can be prepared. In accordance with such a knockout animal, screening for a compound or salt thereof that promotes or inhibits Glis3 promoter activity can be carried out by detecting the level of expression of the reporter gene. Examples of the reporter gene include GFP and other fluorescent protein genes, the lacZ gene, soluble alkaline phosphatase gene, luciferase gene, etc.

Examples of such a transgenic animal and knockout animal include nonhuman mammals such as rabbit, dog, rat, mouse, cow, pig, goat, hamster, etc. Examples of a knockout animal and gene-substituted animal include the aforementioned nonhuman mammals, and among these the mouse is preferred. As the knockout animal and gene-substituted animal, the medaka or another small fish used in research and testing is preferred, and the cells of zebra fish, medaka, goldfish, loach (Misgurnus anguillicaudatus), fugu puffer fish (Takifugu rubripes), etc., are even more preferred. Among the above, fish belonging to the genus Oryzias and related genera, including the transparent medaka disclosed in Japanese Patent Application Laid-open No. 2001-328480, are suitable for various model fish, etc.

Furthermore, prior art, publicly known methods can be used for the methods of configuring a vector and a targeting vector to construct a transgenic animal, knockout animal, and gene-substituted animal, and for the methods of inserting DNA into nonhuman animal ES cells, unfertilized eggs, fertilized eggs, primordial germ cells, etc.

Additionally, the present invention makes it possible to obtain a knockdown animal wherein the endogenous Glis3 gene is inactivated. An antisense nucleic acid that inhibits DNA transcription or inhibits translation to a protein can be used for inactivation of an endogenous gene. In addition, RNA and DNA based on ribozyme and RNAi means, and RNA and DNA based on caging techniques can also be used for inhibiting the expression of an endogenous gene. Furthermore, an aptamer, peptide nucleic acid, and the like can also be used. Just as in the case of the knockout animal, such a knockdown animal will be useful as a PKD model animal.

### (Screening Method for Drugs for Prevention or Treatment of PKD)

The screening method for drugs according to the present invention enables a compound or salt thereof that can compensate for a deficiency of the GLIS3 protein and be used for the treatment of PKD to be obtained by exposing a test compound to a nonhuman PKD model animal such as a medaka, mouse, etc., or animal cells wherein expression of the Glis3 gene is inhibited, and observing whether the formation of cysts is inhibited in the model animal, or whether the formation of cysts and pathology progress. Moreover, by using microorganisms, animal cells, etc., that have been transformed to express the polynucleotide of the present invention, it is possible to detect a target compound that binds specifically to these transformants or to the transcription product of that polynucleotide. Furthermore, it is possible thereby to detect a protein specifically expressed in such a transformant. Additionally, by synthesizing a protein in a cell-free system using that nucleic acid molecule, it is possible to detect a target compound that binds specifically to that protein. Screening for a drug that can be used to prevent or treat PKD can be carried out by detecting such proteins and low molecular weight compounds.

The protein of the present invention and antibody thereto can be used in the drug screening method of the present invention. By using the protein of the present invention or a part thereof, and screening for substances that bind thereto and substances that regulate the expression and activity thereof, it will be possible to discover drugs that cause the protein of the present invention to be expressed or promote the function thereof. Drugs that promote the expression of the protein of the present invention can be discovered by screening for substances that regulate the expression of the protein of the present invention with ELISA and flow cytometry utilizing the antibody of the present invention. A substance that causes the protein of the present invention to be expressed or that enhances the activity thereof can be expected to provide a novel drug for PKD, including ADPKD.

### (Drug for Prevention or Treatment of PKD)

The drug for the prevention or treatment of PKD according to the present invention can contain the DNA of the present invention, and preferably is a vector configured to express the GLIS3 protein or a part thereof. The drug can also contain a suitable base substance for a gene therapy drug. More specifically a vector containing a base sequence encoding cDNA of the human Glis3 gene is a preferred gene therapy drug for the prevention or treatment of human PKD. This gene therapy drug can cause expression of the Glis3 gene in a PKD patient, can inhibit or circumvent the onset of ADPKD and other forms of PKD by producing the protein as the product thereof, and can control the pathological progression of PKD, or can cure PKD. In addition, the drug for the prevention or treatment of PKD according to the present invention can contain the polypeptide of the present invention or part thereof, or an antibody thereto. With such a drug it will be possible to supplement the deficiency of the GLIS3 protein or part thereof and impart the original function of the GLIS3 protein. Such a drug can contain one or a plurality of pharmacologically acceptable carriers.

### EXAMPLE 1

The present invention is described in detail below through examples, but the present invention is by no means limited thereto.

### (Medaka Phylogenetic Analysis)

A phylogenetic analysis (846 individuals) was carried out using sibling mating between the progeny of the medaka pc mutant (hereinafter, also referred to as pc), which is the medaka strain wherein PKD occurs, and progeny of the HNI(+/+) inbred strain. The BAC gene library used for screening originated in the Hd-rR (+1+) inbred strain. This BAC gene library was obtained from professor Hiroshi Hori, Graduate School of Science, Nagoya University.

### (Genetic Analysis)

After the BAC DNA was purified by a conventional mini-prep protocol, the BAC terminal was directly sequenced and mapped. After BAC174E15 was sheared using a HydroShear, the small DNA fragments were fractionated and removed using a Sep 400 Spun Column/Sepharose CL-4B (Amersham), and subcloned with PUC18 plasmids. A BAC174E15 shotgun library was prepared by inserting the plasmids into E. coli DH1OB cells using electroporation. Colonies were randomly selected and sequenced using an Applied Biosystems Model 377 Automated DNA Sequencer.

### (Polymorphism Analysis)

By using 9% polyacrylamide gel electrophoresis (PAGE), it was verified whether or not the DNA fragments of different sizes would be amplified by PCR using genomic DNA of the medaka HNI strain, pc strain, and a crossbred strain thereof. When no size differences were found among the above 3 strains, each PCR product was additionally subjected to a restriction enzyme treatment, and the differences in the band patterns of the products thereof were investigated by PAGE in the same manner.

### (Mutation Analysis)

Total RNA isolated from adult medaka kidney was subjected to reverse transcription, and single-stranded cDNA was prepared thereby. Genomic DNA was prepared from adult medaka caudal fin by conventional methods. RT-PCR of the pc allele and PCR of the genome were performed using the following parameters: 30 cycles of 30 sec at 95° C., 30 sec at 60° C., and 1 to 6 min at 72° C.

(Cloning of medaka pc cDNA)

A RACE library was prepared using a Marathon cDNA Amplification Kit (BD Biosciences) with poly+RNA isolated from adult kidney of the pc strain (−/−) and the OR strain (+/+). Using a primer complementary to the anchor sequence and a primer specific to the pc gene, two rounds of 5′ RACE and 3′ RACE PCR were performed with each nest procedure. The PCR parameters were as follows: 30 cycles of 30 sec at 95° C., 30 sec at 62° C., and 3 min at 72° C. The base sequences of the gene-specific primers used for 5′ RACE and 3′ RACE are shown in the following table.

TABLE 1

| | | |
|---|---|---|
| 5′-cagcatcttcctgga | (ex2-31, | (SEQ ID NO: 9) |
| ctgtgg-3′ | 5′ RACE | |
| | first round) | |
| 5′-cacattcgaactgtg | (ex2-32, | (SEQ ID NO: 10) |
| tggctgg-3′ | 5′ RACE | |
| | second round) | |
| 5′-tttgaaggctgcaag | (c67-F, | (SEQ ID NO: 11) |
| aaggcatt-3′ | 3′ RACE | |
| | first round) | |
| 5′-ctcgacttgaaaacc | (c67-F3, | (SEQ ID NO: 12) |
| tgaagat-3′ | 3′RACE | |
| | second round) | |

After the RACE PCR products were subcloned to pDrive cloning vectors (QIAGEN), they were sequenced using an Applied Biosystems Model 377 Automated DNA Sequencer.

### (Mouse and Human pc Homologues)

A publicly accessible database was searched using the tBlastn program, and proteins having a high level of homology with the medaka pc protein amino acid sequence were identified.

### (Expression Analysis)

Northern blotting was carried out by conventional methods using total RNA prepared from adult kidney of the pc strain and OR strain. A set of sense and antisense primers specific to the genes to be studied (including pc) was used for RT-PCR. Whole-mount in situ hybridization was performed wherein a DIG-labeled pc riboprobe was hybridized with the OR strain sample.

### (Results)

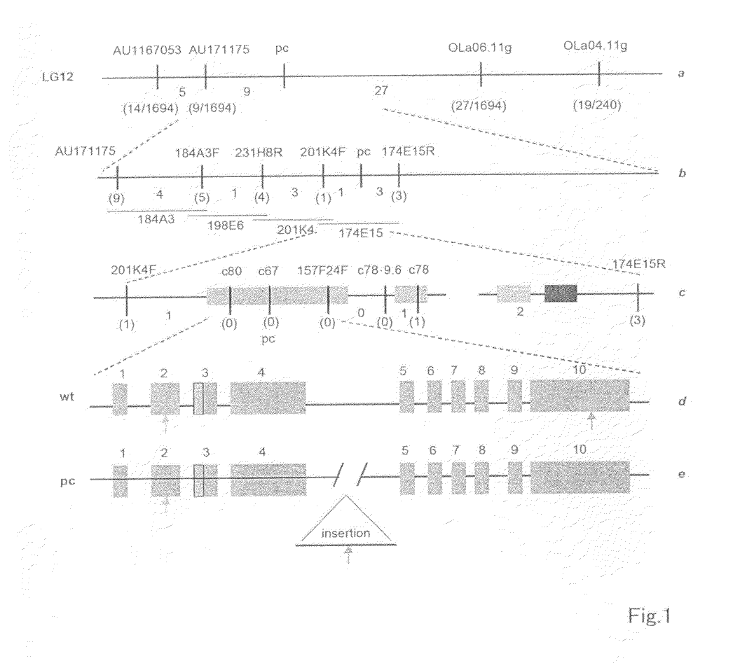

M-marker 2003 (<http://medaka.lab.nig.ac.jp/mmarker.htm>), a publicly accessible database, was used with a bulked segregant analysis tool, and it was found that the pc gene locus lies in linkage group 12. In addition, as shown in FIG. 1, from the results of a linkage group analysis by known polymorphism markers mapped in linkage group 12, the pc gene locus was mapped to within 0.5 cM (9/847×2) of AU171175 and within 1.6 cM (27/847×2) of OLa06.11g.

Because there was no existing polymorphism marker at a position closer to the pc gene locus than polymorphism marker AU171175, chromosome walking was carried out from the AU171175 position using BAC. In the first walk BAC184A3 was closest to the pc gene locus, and the number of recombination between polymorphism marker 184A3F (SEQ ID NOs: 13 and 14) on the BAC clone and the pc gene locus was 5/847×2. In the second walk, BAC198E6 was closest to the pc gene locus, and the number of recombination between polymorphism marker 231H8R (SEQ ID NOs: 15 and 16) on the BAC clone and the pc gene locus was 4/847×2. Likewise, in the third walk the number of recombination between polymorphism marker 201 K4F (SEQ ID NOs: 17 and 18) on the BAC clone, which was identified based on the most proximal BAC201K4, and the pc gene locus was 1/847×2. In the fourth walk BAC174E15, recognized as most proximal, had polymorphism marker 174E15R (SEQ ID NOs: 19 and 20) on its terminus, and that polymorphism marker was mapped to within 0.2 cM (3/847×2) on the opposite side of the starting position of chromosome walking from the viewpoint of the pc gene locus. From this finding, it was predicted that BAC174E15 must straddle the region of the pc gene locus (FIG. 1).

When shotgun sequencing of BAC174E15 was carried out, the BAC DNA was linked to a total of 7 scaffolds comprising 15 contigs. These scaffolds can be aligned by comparisons with homologous regions in the genome, and the base sequences indicated that BAC174E15 contains 5 genes. These 5 genes were homologues of genes lying in a homologous region of the fugu genome. As shown in FIG. 1, because the respective numbers of recombination between the pc gene locus and c80 (SEQ ID NOs: 23 and 24), c67-1 (SEQ ID NOs: 25 and 26), 157F24F (SEQ ID NOs: 21 and 22), and c78 (SEQ ID NOs: 27 and 28), which are polymorphism markers identified in two of these genes, were 0/847×2, 0/847×2, 0/847×2, and 1/847×2, it was assumed that the pc gene locus was present in the region of these two genes.

The pc gene was thought to be a gene in the region containing c80, c67 and 157F24F, or a gene containing c78, and the results of a homology analysis showed that these genes have a high level of homology with the human genes encoding the Glis3 (Gli-similar3) and RFX3 (regulatory factor X3) proteins, respectively. Glis3 is a transcription factor having five C2H2 zinc fingers, but its function in humans and mice is unknown. RFX3 is known to be a transcription factor regulating the expression of HLA Class II genes.

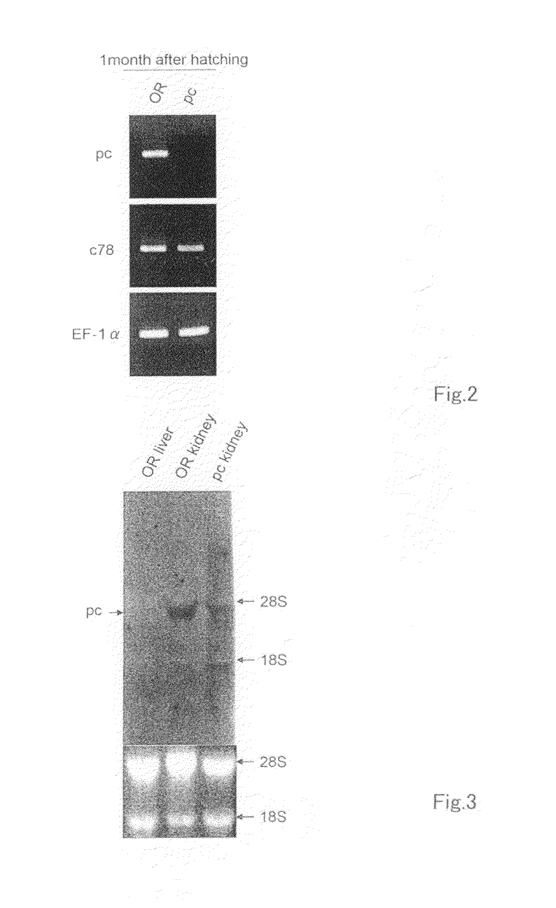

Expression of mRNA of RFX3 and the region homologous to Glis3 was investigated by RT-PCR using primer set c67 (c80-67, SEQ ID NOs: 29 and 30) and primer set c78 (SEQ ID NOs: 27 and 28). As shown in FIG. 2, the mRNA of both was detected in the kidney of the wild type medaka OR(+/+). Although RFX3 mRNA was detected in the pc (−/−) kidney, Glis3 mRNA was not. Additionally, as shown in FIG. 3, expression of Glis3 was confirmed in OR (+1+) kidney by northern blotting using the PCR product of c80 as a probe, however, the expression was hardly confirmed in the pc (−/−) kidney.

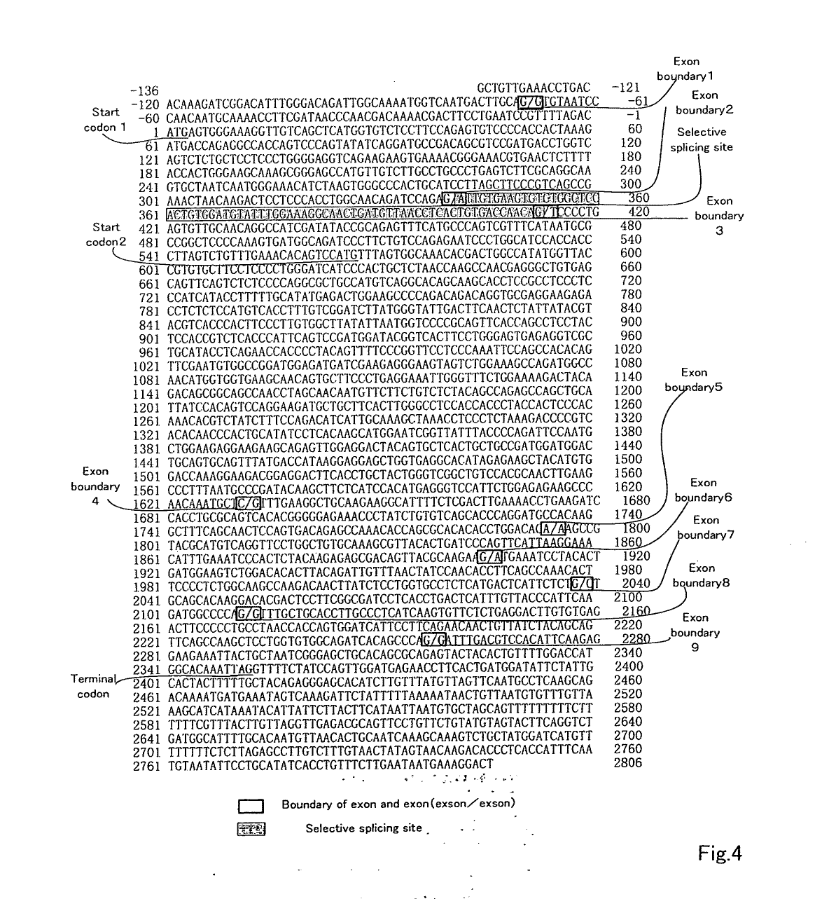

The whole ORF of medaka Glis3 was sequenced by 5′ RACE and 3′ RACE, and the boundaries between exons and introns were determined. Selective splicing involving different lengths for exon 1 and exon 3, respectively, was found in the medaka Glis3 gene, and these encoded at least 2 forms of the GLIS3 protein (SEQ ID NOs: 1 and 3, FIG. 4) estimated to comprise 783 amino acids (pc-a) (SEQ ID NO: 2) and 595 amino acids (pc-b) (SEQ ID NO: 4).

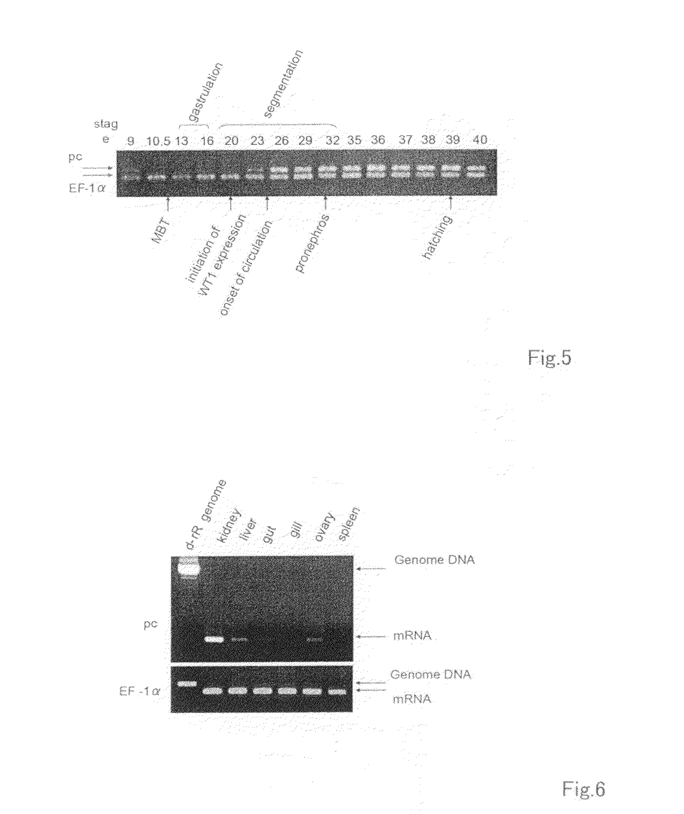

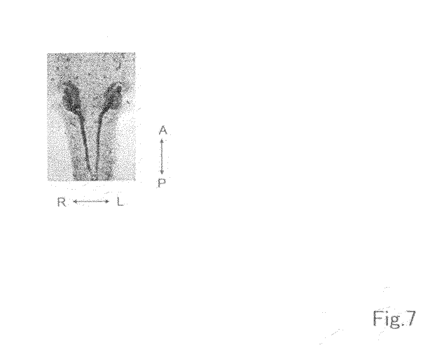

As shown in FIG. 5, when pc gene expression was investigated by RT-PCR, maternal mRNA was detected at stage 9, and at one time (stages 10.5 to 20) the mRNA was undetectable, but the mRNA began to be detected about stage 23. Throughout embryo development pc mRNA was continuously expressed at a detectable level. As shown in FIG. 6, strong expression of pc mRNA in adult kidney was found using the same method. The same mRNA was detected in liver and ovary, but only in trace amounts. Additionally, as shown in FIG. 7, by whole-mount in situ hybridization expression of pc mRNA in the renal tubules and mesonephric duct was found in fry on days 5, 10 and 20 post-hatching.

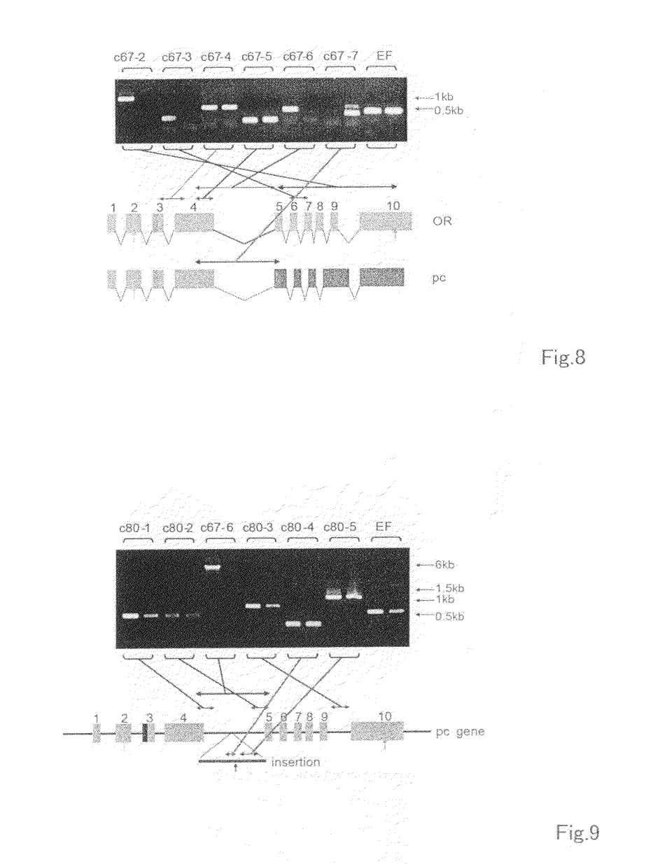

As shown in FIG. 8, the expression of pc mRNA in medaka adult kidney was investigated using pc-specific primer sets (shown in FIG. 8: c67-2 (SEQ ID NOs: 31 and 32), c67-3 (SEQ ID NOs: 33 and 34), c67-4 (SEQ ID NOs: 35 and 36), c67-5 (SEQ ID NOs: 37 and 38), c67-6 (SEQ ID NOs: 39 and 40), c67-7 (SEQ ID NOs: 41 and 42), and EF (SEQ ID NOs: 43 and 44)). Although the fragment corresponding to the 5′ side of exons 3-4 was amplified in the pc mutant, the fragment on the 3′ side of exon 5 was not amplified. On the other hand, in the OR (+/+) strain, the pc gene was amplified by all the primer sets. From these findings, it was predicted that in the pc mutant the region on the 3′ side of exon 4 of the pc gene is missing.

Moreover, as shown in FIG. 9, the genomic regions on the 5′ side of exon 4 and the genomic regions on the 3′ side of exon 5 were amplified by primer sets specific to each: c80-1 (SEQ ID NOs: 45 and 46), c80-2 (SEQ ID NOs: 47 and 48), c67-6 (SEQ ID NOs: 39 and 40), c80-3 (SEQ ID NOs: 49 and 50), c80-4 (SEQ ID NOs: 51 and 52), c80-5 (SEQ ID NOs: 53 and 54), and EF (SEQ ID NOs: 43 and 44). The genomic fragment covering exons 4 and 5 was about 6 kb long in the OR strain, but no amplification thereof was seen in the pc mutant.

From these findings, it is thought this genomic fragment was not amplified by PCR in the pc mutant because there is some kind of insertion in the intron between exons 4 and 5, and the distance between the two exons has become much larger than 6 kb.

The region on the 5′ side of exon 4 was detected as mRNA in the pc mutant. On the other hand, the 3′ side of exon 5 was not detected as mRNA. Therefore, to investigate what kind of structure is present on the 3′ side of exon 4, 3′ RACE was carried out using primers ex62-3-52 (SEQ ID NO: 35) and c80-F2 (SEQ ID NO: 29). At present it is still unknown whether the 3′ region of pc mRNA obtained from the pc mutant is one that originally should be transcribed as part of some gene, but at least 5 exons having sequences other than that of the pc gene are attached on the 3′ side of exon 4 of the pc gene (see FIG. 4). Furthermore, the transcription product of the 3′ region varies, and several of these 5 exons are randomly dropped. The DNA sequence seen in the 3′ region of pc mRNA from the pc mutant was not specific to the pc mutant, and was also present in the genome of medaka strain d-rR (+1+) (FIG. 9).

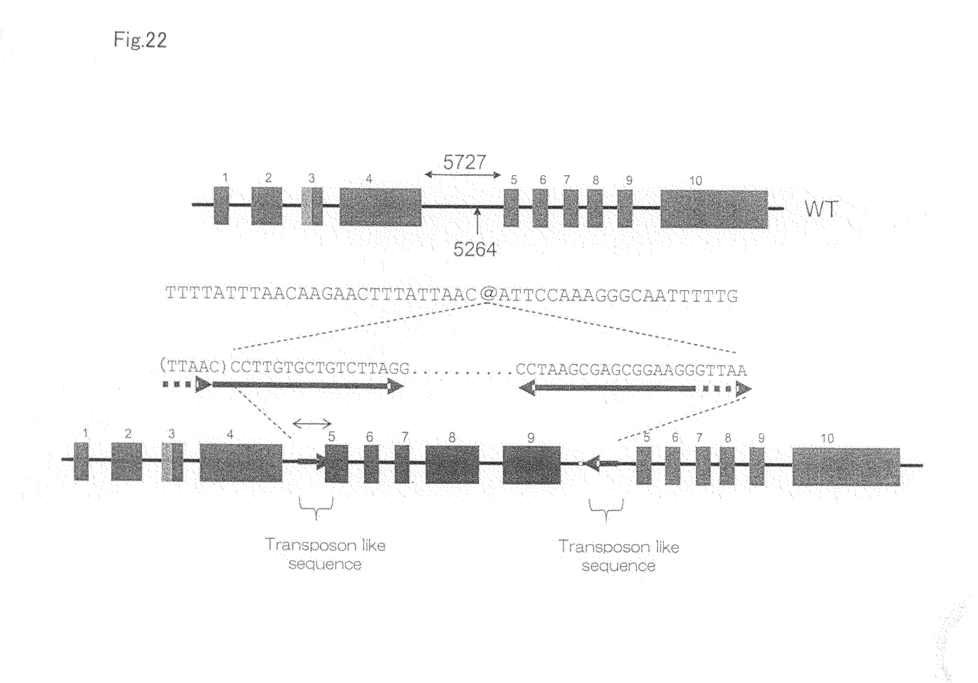

The base sequence of the specifically amplified PCR fragment (both arrows, lower row of FIG. 22) between intron 4 and exon 5 (indicating an exon specific to the pc mutant) on the pc mutant genome was compared with the base sequence of the wild type. Intron 4 of the wild type pc gene consists of 5727 bases, but a specific insertion sequence between base 5264 and base 5265 was seen in the mutant. This insertion sequence is no less than 10 kb long, and on both ends a repeat sequence of four bases (TTAA) and an inverted repeat sequence of 18 bases (CCCTTGTGCTGTCTTAGG) were found. Furthermore, approximately 300 bases at both ends of the insertion sequence are essentially the same as a transposon seen at the mutation site of medaka mutant rs-3, which has already been identified as the causative gene of a scale abnormality (Curr Biol. 7 Aug. 2001; 11(15): 1202-6). Additionally, it was found that the sequence on the 3′ side of exon 5 in the pc mRNA of the mutant corresponds to part of the insertion sequence. From these findings it was assumed that in the pc mutant an endogenous DNA sequence has translocated to between exons 4 and 5 of the pc gene.

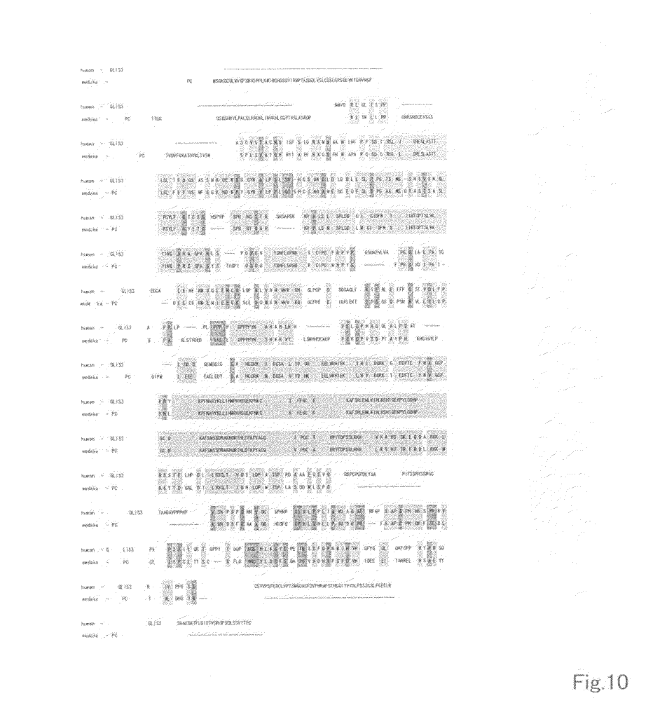

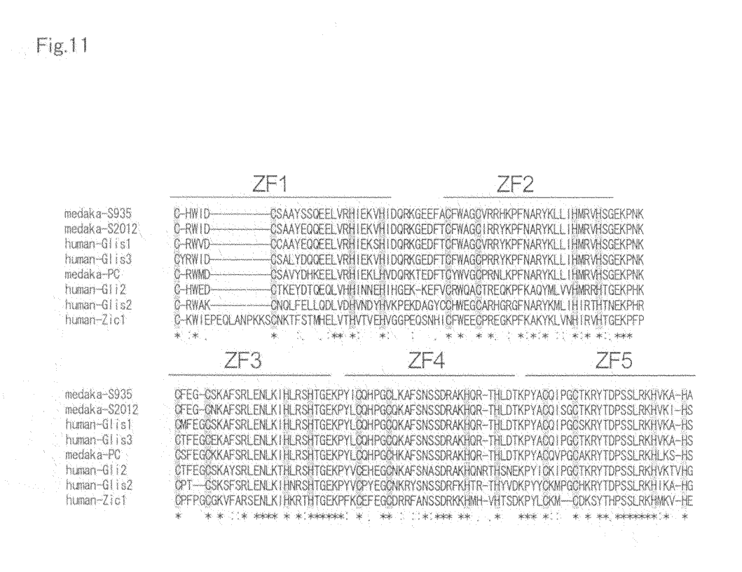

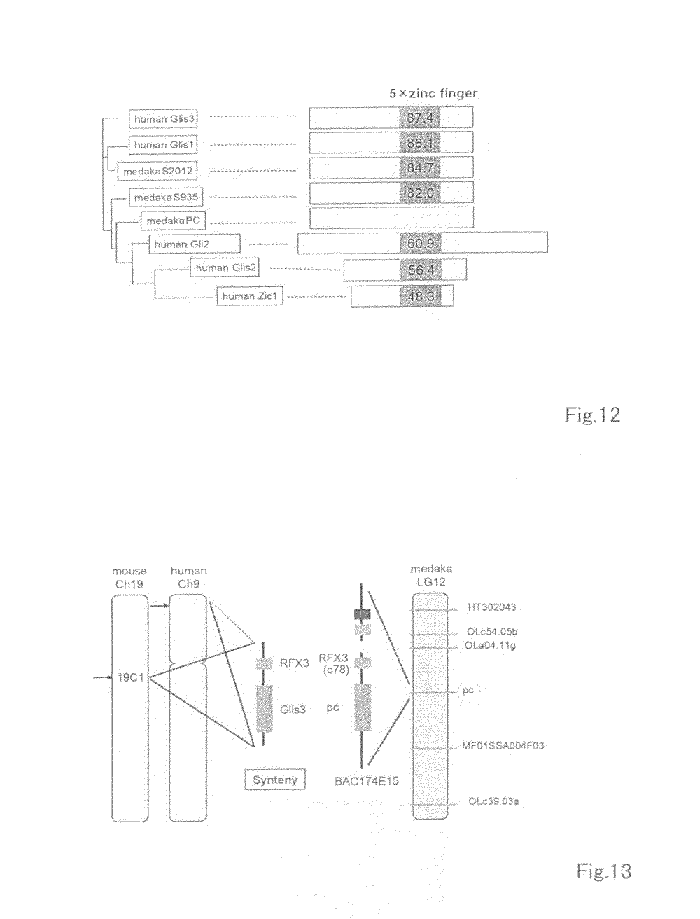

As shown in FIG. 10, the pc protein is a transcription factor having five C2H2 zinc fingers. The pc amino acid sequence is 48% (pc-b: 49%) identical to the human Glis3 protein and 50% (pc-b: 52%) identical to the mouse Glis3 protein. In particular, as shown in FIGS. 11 and 12, there is a high level of similarity with Glis in the zinc finger regions, and this is significantly greater than the similarity thereto of other Gli and Zic, which also are related members of this zinc finger family. In humans, the GLIS protein exists as at least 3 different molecules, GLIS 1, GLIS2, and GLIS3.

As shown in FIG. 13, it was noted above that the medaka pc gene is adjacent to the RFX3 gene, and in humans and mice the Glis3 gene and RFX3 gene are in adjacent positions on chromosome 9 (9p24.2) and chromosome 19 (19C1). From such synteny, it is believed that the medaka pc gene is a homologue of the human and murine Glis3 gene.

Proceeding to identify the function of the gene of the present invention will enable a major leap in elucidating the whole picture concerning the onset of PKD in humans, which heretofore has been unknown. In addition, it will also contribute to the development of drugs for the treatment of PKD focusing on the function of the gene of the present invention.

### EXAMPLE 2

(Medaka Kidney in situ Hybridization using a pc RNA Probe)
(1) Expression of pc mRNA in Wild Type Medaka

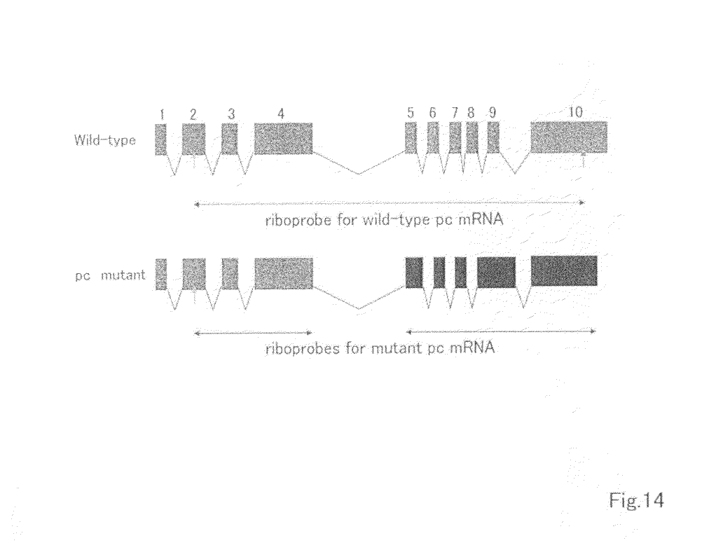

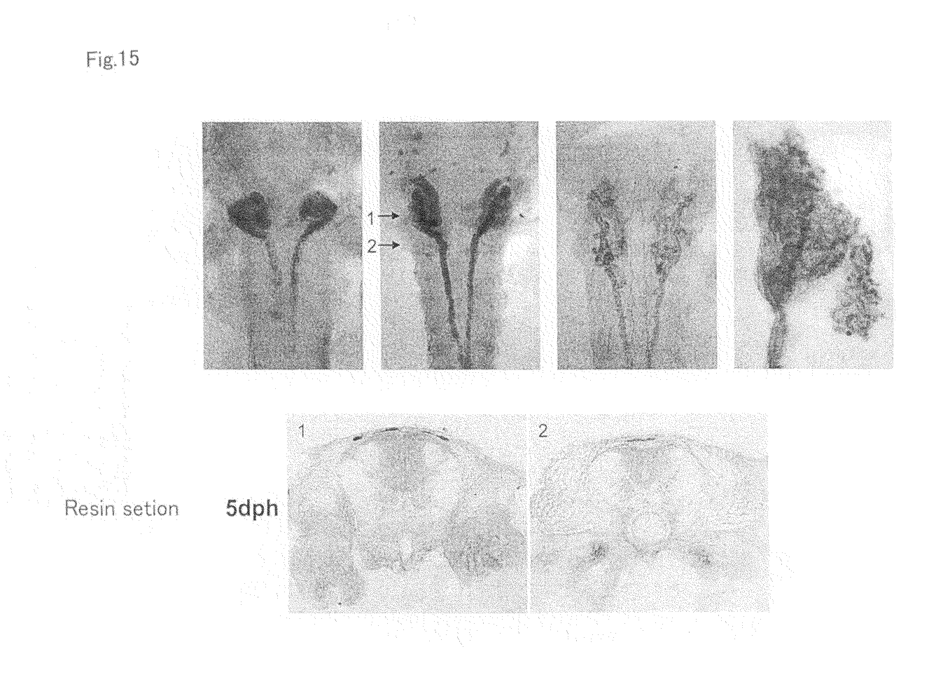

After the gut and other visceral organs were removed by necropsy, whole-mount in situ hybridization of the kidneys was carried out by conventional methods. As shown in the top row of FIG. 14, a DIG-labeled RNA probe (riboprobe) was synthesized by SP6 RNA polymerase in the presence of DIG-11-UTP using a plasmid vector containing the whole open reading frame of the pc cDNA as a template. For observation of the tissues wherein it was expressed, the collected kidneys were embedded in Technovit 8100™ and cut into 10μ sections with a microtome. The cell nuclei were stained with neutral red for visual identification of the sites where pc mRNA was not expressed. The top row of FIG. 15 shows images taken from the ventral side of pre-necropsy medaka at 0, 5, 20 and 30 days after hatching, and the bottom row shows images taken of sections prepared from a medaka at 5 days after hatching. The arrows on the top row in the figure indicate the location of each section in the bottom row. As shown in FIG. 15, a signal specific to the epithelial cells of the renal tubules and urinary duct was seen.

(2) Expression of pc mRNA in pc Mutant (pcST)

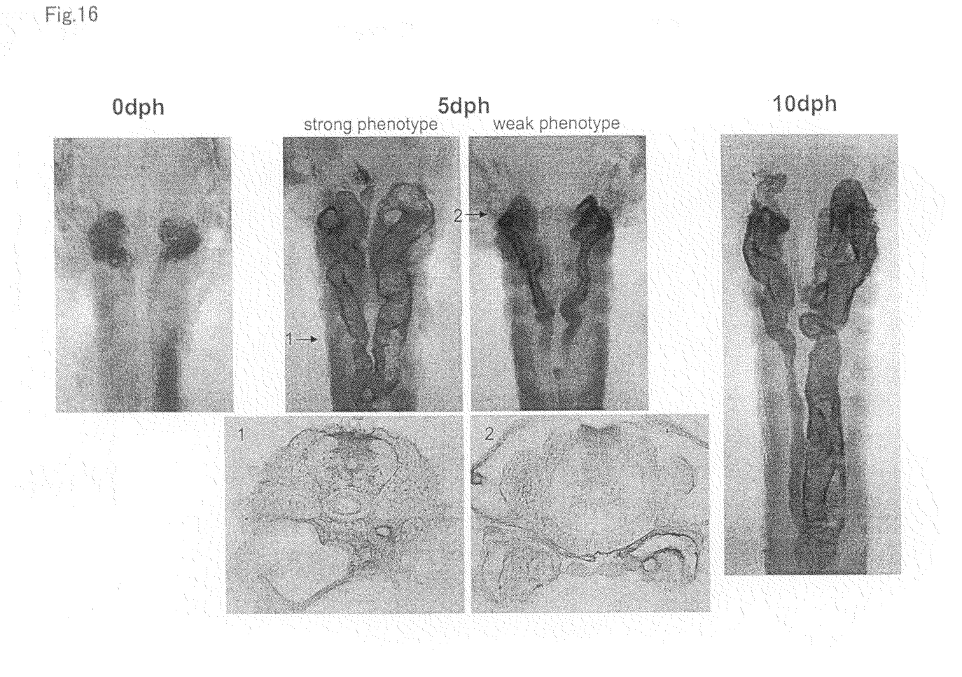

As shown in the bottom row of FIG. 14, riboprobes were synthesized from cDNA fragments corresponding to the regions of exons 2-4 (same part as in the wild type) and to the 3′ region seen only the mutant, and these were mixed together and used. Samples were prepared using the same procedure as described in (1), and they were stained to enable visual identification. The top row of FIG. 16 shows images taken from the ventral side of pre-necropsy medaka at 0, 5, and 10 days after hatching, and the bottom row shows images of sections prepared from the medaka at day 5 after hatching. The sections in the bottom row each correspond to in situ hybridization samples in the top row, and the arrows in the top row show the position of each section in the bottom row. As shown in FIG. 16, the pc gene was expreised in the pc mutant. However, the normal mRNA region extends to exon 4, and thereafter a sequence originating in the huge insertion fragment inserted in intron 4 is attached (see FIG. 14). The site of expression of this abnormal mRNA is specific to the epithelial cells of the renal tubule and urinary duct just as in the wild type. Thus, enlargement of the tubules and ducts in the pc mutant could be seen, and the formation of cysts in the kidneys was clearly visible through the expression of the pc gene per se.

### EXAMPLE 3

(Knockdown using Antisense Oligonucleotide)

### (1) Preparation of Knockdown Medaka

GripNA from Active Motif, Inc. was used for the antisense oligonucleotides. The oligonucleotides were designed to form complementary strands to the 18 bases covering the start codons of the pc gene. By such a design the oligonucleotides could be expected to specifically inhibit the translation of pc mRNA. Because it is believed that 2 types of mRNA are produced from the pc gene due to a difference in splicing, and each is translated from a different start codon, the following antisense oligonucleotides were prepared: pcaNA=5′-CACTCATGTCTAAAACGG-3′ (SEQ ID NO: 55) and pcbNA=5′-ACTAAACATGGACTGTGT-3′ (SEQ ID NO: 56). Embryos inserted with approximately 0.5 ng of each by microinjection at cell stages 1 to 4 were raised to 0 to 5 days after hatching. Then paraffin sections were prepared, and the kidneys were examined.

### (2) Knockdown Statistics

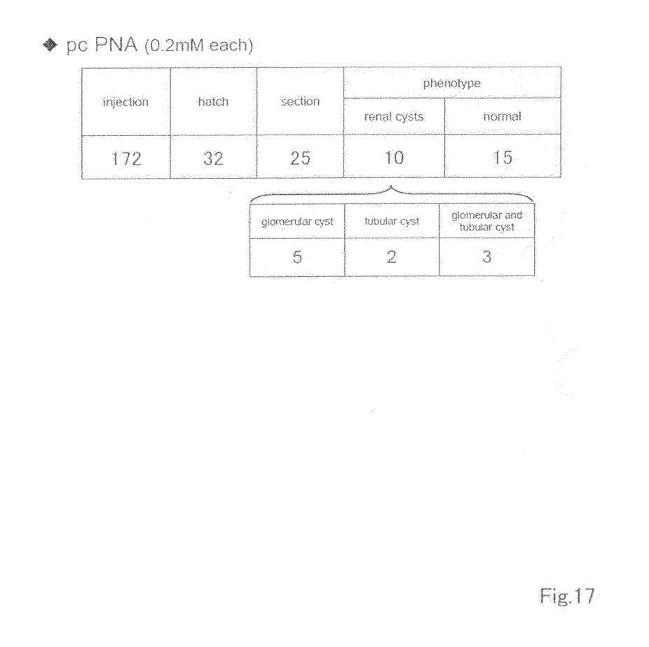

FIG. 17 shows the statistics related to the knockdown. As shown in FIG. 17, after the antisense oligonucleotides were inserted into 172 embryos by microinjection, 32 actually hatched. From the hatched individuals, 25 were randomly selected and sections were prepared. In the sections of the 25 individuals, some with cyst formation in the glomerulus (Bowman's capsule), some with cyst formation in the renal tubules or urinary duct, and some with cyst formation in both were found. When these are combined, cysts were observed in 40% of the individuals (10 of the 25 individuals from which sections were prepared).

(3) Phenotype Observations from Tissue Sections

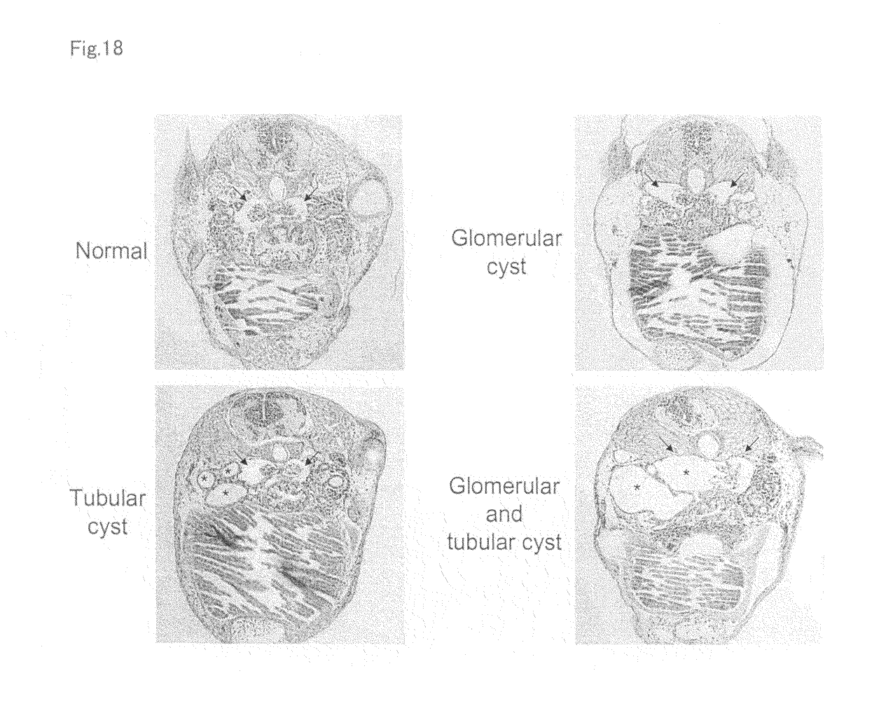

Various knockdown individuals (fry) were fixed in the conventional manner by Bouin fixation, 6 μm paraffin sections of sites containing glomeruli were prepared, and these were stained with hematoxylin-eosin stain. The results are shown in FIG. 18. As shown in FIG. 18, in comparison with the wild type medaka, the lumen size in the glomerulus (Bowman's capsule), renal tubule or urinary duct, or both is markedly enlarged in the knockdown individuals. In other words, in comparison with the others, in the individuals on the top right and bottom right the Bowman's capsule part of the glomerulus indicated by the arrows clearly accounts for the large lumen. In the individuals on the bottom left and right, conspicuously enlarged lumina can be seen in parts of the renal tubules beside the glomerulus (in the figure indicated by asterisks (*) on the left side of both photos).

### EXAMPLE 4

### (1) Preparation of Double Knockdown Individuals

An antisense oligonucleotide (GripNA) to S2012, S2012-grip=5′-TTTACTCACCATACACTT-3′, was designed to form a complementary strand to the 18 bases covering the splicing donor site (a site corresponding to the 3′ end of exon 4 in the pc gene). The S2012-grip could be expected to inhibit splicing immediately behind the second of the 5 zinc fingers. In the same manner as above, approximately 0.5 ng of S2012-grip was inserted into embryos together with approximately 0.5 ng of pcaNA and approximately 0.5 ng of pcbNA, which are the antisense oligonucleotides used in Example 3, and the phenotypes were observed.

### (2) Double Knockdown Statistics

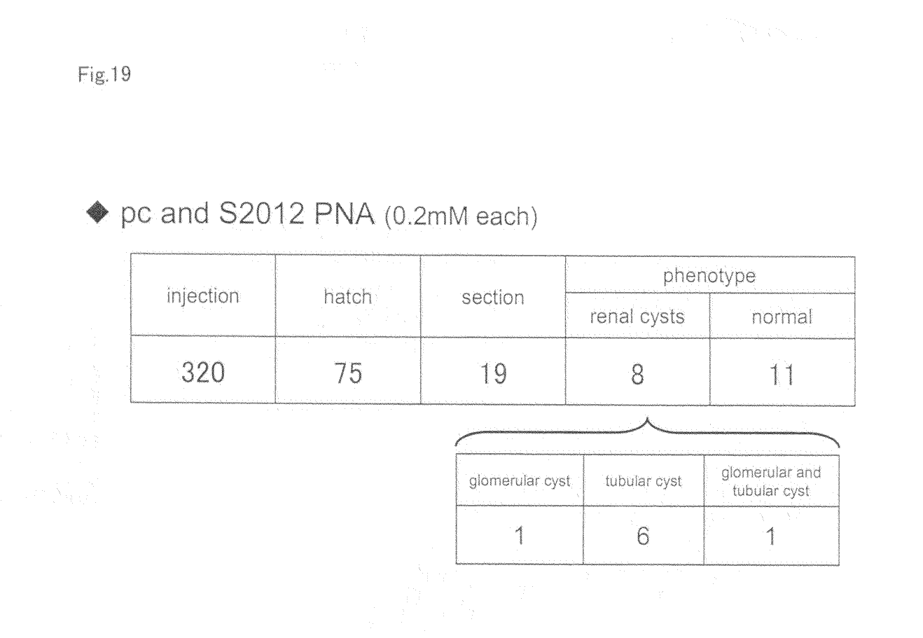

FIG. 19 shows the statistics for the double knockdown. As shown in FIG. 19, after the 3 types of antisense oligonucleotides were inserted into 320 embryos by microinjection, 75 individuals hatched. From among the hatched individuals 19 were selected, and sections were prepared. In the sections of the 19 individuals, some with cyst formation in the glomeruli (Bowman's capsule), some with cyst formation in the renal tubules or urinary duct, and some with cyst formation in both were found. In the double knockdown, some individuals had such large cysts at hatching that they could be distinguished under a dissecting microscope.

(2) Phenotype Observations from Tissue Sections

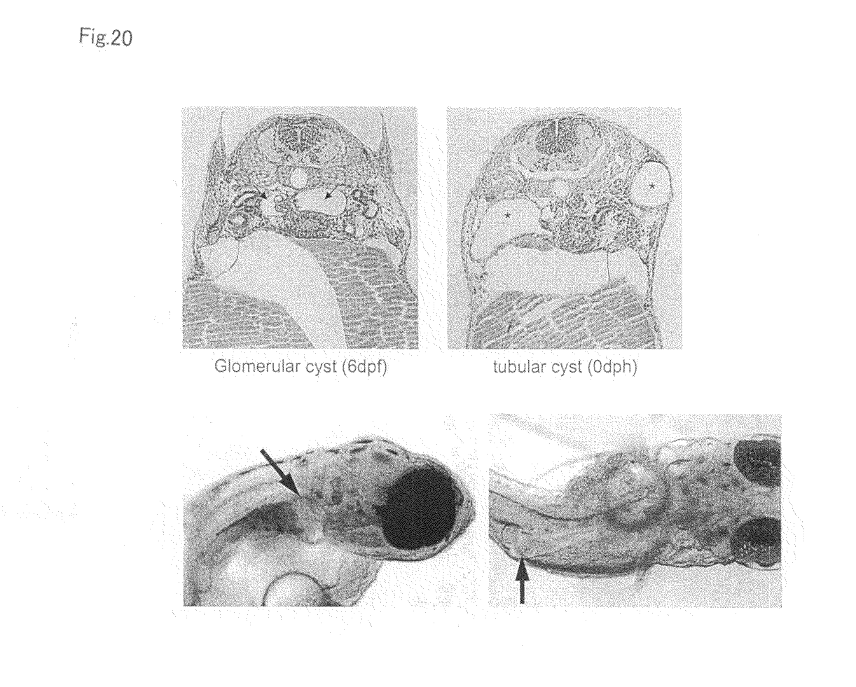

As in Example 3, the tissues were observed in the double knockdown individuals. The results are shown in FIG. 20. As shown in FIG. 20, the cysts are clearly larger than in the pc gene single knockdown. However, reproducibility of this phenotype was poor, and cysts could not be seen in the tissue sections of individuals in which cysts could not be distinguished under a dissecting microscope.

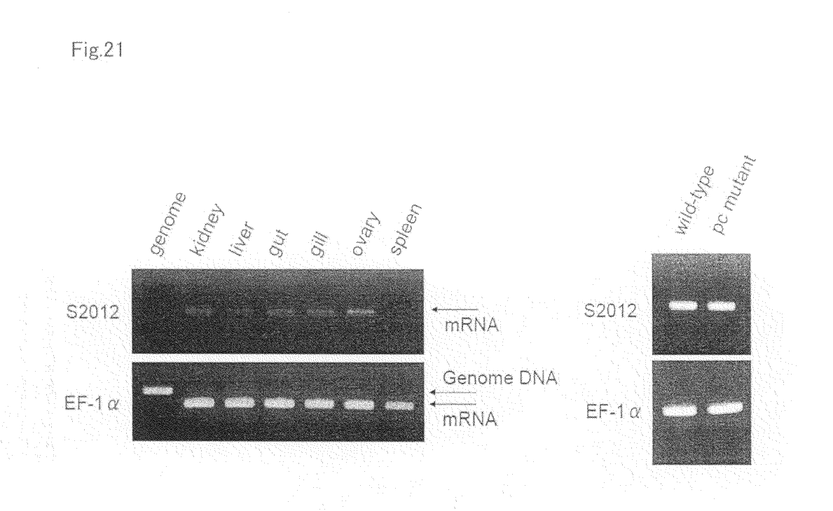

The mouse orthologue of pc is glis3. In mice glis1 has also been isolated as a member of the glis family. When the medaka genome database was searched, S2012 (provisional name), a medaka orthologue of glis 1 was identified (FIG. 11). As shown in FIG. 21, S2012 (glis1) was expressed in all organs except the spleen in medaka, as far as could be investigated.

1. DNA selected from:
(a) DNA encoding a protein having the amino acid sequence of SEQ ID NO: 2 or 4;
(b) DNA encoding a protein having an amino acid sequence in which one or a plurality of amino acid residues is substituted into, deleted from, and/or added to the amino acid sequence of SEQ ID NO: 2 or 4, and having substantially the same activity thereof;
(c) a nucleic acid molecule having the base sequence of SEQ ID NO: 1 or 3; and
(d) DNA that hybridizes under stringent conditions with DNA having a base sequence complementary to the base sequence of SEQ ID NO: 1 or 3.

2. DNA encoding a part of the protein described in (a) or the protein described in (b) according to claim 1.

3. DNA that hybridizes with the nucleic acid molecule according to claim 1, or a complementary strand thereof.

4. DNA having a sequence identical to a continuous base sequence of 100 or fewer bases of the DNA according to claim 1, or a complementary strand thereof.

5. A detection agent containing the DNA according to claim 1.

6. Antisense DNA to the DNA according to claim 1.

7. A vector containing the DNA according to claim 1.

8. The vector according to claim 7, containing RNA equivalent to the DNA sense strand as the DNA.

9. A transformant having the vector according to claim 7 inserted therein.

10. A transformant in which expression of the endogenous DNA according to claim 1 is inhibited.

11. A polypeptide that is either a protein or part thereof selected from a group consisting of:
(a) a protein having the amino acid sequence of SEQ ID NO: 2 or 4; and
(b) a protein having an amino acid sequence in which one or a plurality of amino acid residues is substituted into, deleted from, and/or added to the amino acid sequence of SEQ ID NO: 2 or 4, and having substantially the same activity thereof.

12. An antibody to the polypeptide according to claim 11.

13. A detection agent containing the antibody according to claim 12.

14. A drug for prevention or treatment of polycystic kidney disease containing DNA encoding a GLIS3 protein or a part thereof.

15. The drug according to claim 14, wherein the DNA encoding the GLIS3 protein is DNA selected from the following group:
(a) DNA encoding a protein having any of the amino acid sequences selected from the amino acid sequences of SEQ ID NOs: 2, 4, 6, and 8;
(b) DNA encoding a protein having an amino acid sequence in which one or a plurality of amino acid residues is substituted into, deleted from, and/or added to any of the amino acid sequences selected from the amino acid sequences of SEQ ID NOs: 2, 4, 6, and 8, and having substantially the same activity thereof;
(c) DNA having any of the base sequences selected the base sequences of SEQ ID NOs: 1, 3, 5, and 7; and
(d) DNA that hybridizes under stringent conditions with a base sequence complementary to any of the base sequences selected the base sequences of SEQ ID NOs: 1, 3, 5, and 7.

16. A method for prevention or treatment of polycystic kidney disease comprising a step of administering an effective amount of DNA encoding a GLIS3 protein or part thereof to an animal.

17. A diagnostic agent for polycystic kidney disease containing DNA encoding a GLIS3 protein or a part thereof, or antisense DNA having a base sequence that is complementary to or substantially complementary to the base sequences of those DNA molecules.

18. A method for diagnosis of polycystic kidney disease containing DNA encoding a GLIS3 protein or a part thereof, or antisense DNA having a base sequence that is complementary to or substantially complementary to the base sequences of those DNA molecules.

19. A screening method for a drug for prevention or treatment of polycystic kidney disease that utilizes DNA encoding a GLIS3 protein or a part thereof, or antisense DNA having a base sequence that is complementary to or substantially complementary to the base sequences of those DNA molecules.

20. A screening method for a drug for prevention or treatment of polycystic kidney disease using cells that express a Glis3 gene.

21. A screening method for a drug for prevention or treatment of polycystic kidney disease using cells in which expression of a Glis3 gene is inhibited.

22. The screening method for a drug for prevention or treatment of polycystic kidney disease according to claim 21, using a transformant animal in which expression of a Glis3 gene is inhibited.

23. A drug for prevention or treatment of polycystic kidney disease containing a GLIS3 protein, a part thereof, or a salt thereof.

24. The drug according to claim 23, wherein the GLIS3 protein is:
(a) a protein having any of the amino acid sequences selected from the amino acid sequences of SEQ ID NOs: 2, 4, 6, and 8; or
(b) a protein having an amino acid sequence in which one or a plurality of amino acid residues is substituted into, deleted from, and/or added to any of the amino acid sequences selected from the amino acid sequences of SEQ ID NOs: 2, 4, 6, and 8 and having substantially the same activity thereof.

25. A method for prevention or treatment of polycystic kidney disease comprising a step of administering an effective amount of a GLIS3 protein, a part thereof, or a salt thereof to an animal.

26. A screening method for a drug for prevention or treatment of polycystic kidney disease using a GLIS3 protein, a part thereof, or a salt thereof.

27. A screening method for a drug for prevention or treatment of polycystic kidney disease, using cells capable of expressing a GLIS3 protein or a part thereof.

28. A diagnostic agent for polycystic kidney disease containing an antibody to a GLIS3 protein, a part thereof, or a salt thereof.

29. A diagnostic method for polycystic kidney disease utilizing an antibody to a GLIS3 protein, a part thereof, or a salt thereof.


SEQUENCE LISTING (56 sequences)

SEQ ID NO: 1 - DNA, 2349 bp, Oryzias latipedes
  CDS (1)..(2349)
atgagtgggaaaggttgtcagctcatggtgtctccttccagagtgtccccaccactaaagatgaccagaggccaccagtcccagtatatcaggatgccgacagcgtccgatgacctggtcagtctctgctcctccctggggaggtcagaagaagtgaaaacgggaaacgtgaactcttttaccactgggaagcaaagcgggagccatgttgtcttgcctgccctgagtcttcgcaggcaagtgctaatcaatgggaaacatctaagtgggcccactgcatccttagcttcccgtcagccgaaactaacaagactcctcccacctggcaacagatccagagattgtgaagtgtctggctccactgtggatgtatttggaaaggcaactgatgttaacctcactgtgaccaacagtcccctgagtgttgcaacaggccatcgatataccgcagagtttcatgcccagtcgtttcataatgcgccggctccccaaagtgatggcagatcccttctgtccagagaatccctggcatccaccacccttagtctgtttgaaacacagtccatgtttagtggcaaacacgactggccatatggttaccgtgtgcttcctcccctgggatcatcccactgctctaaccaagccaacgagggctgtgagcagttcagtctctccccaggcgctgccatgtcaggcacagcaagcacctccgcctccctcccatcatacctttttgcatatgagactggaagccccagacagacaggtgcgaggaagagacctctctccatgtcacctttgtcggatcttatgggtattgacttcaactctattatacgtacgtcacccacttcccttgtggcttatattaatggtccccgcagttcaccagcctcctactccaccgtctcacccattcagtccgatggatacggtcacttcctgggagtgagaggtcgctgcatacctcagaaccacccctacagttttcccggttcctcccaaattccagccacacagttcgaatgtggccggatggagatgatcgaagagggaagtagtctggaaagccagatggccaacatggtggtgaagcaacagtgcttccctgaggaaattgggtttctggaaaagactacagacagcggcagccaacctagcaacaatgttcttctgtctctacagccagagccagctgcattatccacagtccaggaagatgctgcttcacttgggcctccaccaccctaccactcccacaaacacgtctatctttccagacatcattgcaaagctaaacctccctctaaagaccccgtcacacaacccactgcatatcctcacaagcatggaatcggttatttaccccagattccaatgctggaagaggaagaagcagagttggaggactacagtgctcactgctgccgatggatggactgcagtgcagtttatgaccataaggaggagctggtgaggcacatagagaagctacatgtggaccaaaggaagacggaggacttcacctgctactgggtcggctgtccacgcaacttgaagccctttaatgcccgatacaagcttctcatccacatgagggtccattctggagagaagcccaacaaatgctcgtttgaaggctgcaagaaggcattttctcgacttgaaaacctgaagatccacctgcgcagtcacacgggggagaaaccctatctgtgtcagcacccaggatgccacaaggctttcagcaactccagtgacagagccaaacaccagcgcacacacctggacacaaagccgtacgcatgtcaggttcctggctgtgcaaagcgttacactgatcccagttcattaaggaaacatttgaaatcccactctacaagagagcgacagttacgcaagaagatgaaatcctacactgatggaagtctggacacacttacagattgtttaactatccaacaccttcagccaaacacttcccctctggcaagccaagacaacttatctcctggtgcctctcatgactcattctctgctgcagcacaaggacacgactccttcggcgatcctcacctgactcatttgttacccattcaagatggccccaggtttgctgcaccttgccctcatcaagtgttctctgaggacttgtgtgagacttccccctgcctaaccaccagtggatcattccttcagaacaactgttatctacagcagttcagccaagctcctggtgtggcagatcacagcccaggatttgacgtccacattcaagaggaagaaattactgctaatcgggagctgcacagcgcagagtactacactgttttggaccatggcacaaat

SEQ ID NO: 2 - protein, 783 aa, Oryzias latipedes
MSGKGCQLMVSPSRVSPPLKMTRGHQSQYIRMPTASDDLVSLCSSLGRSEEVKTGNVNSFTTGKQSGSHVVLPALSLRRQVLINGKHLSGPTASLASRQPKLTRLLPPGNRSRDCEVSGSTVDVFGKATDVNLTVTNSPLSVATGHRYTAEFHAQSFHNAPAPQSDGRSLLSRESLASTTLSLFETQSMFSGKHDWPYGYRVLPPLGSSHCSNQANEGCEQFSLSPGAAMSGTASTSASLPSYLFAYETGSPRQTGARKRPLSMSPLSDLMGIDFNSIIRTSPTSLVAYINGPRSSPASYSTVSPIQSDGYGHFLGVRGRCIPQNHPYSFPGSSQIPATQFECGRMEMIEEGSSLESQMANMVVKQQCFPEEIGFLEKTTDSGSQPSNNVLLSLQPEPAALSTVQEDAASLGPPPPYHSHKHVYLSRHHCKAKPPSKDPVTQPTAYPHKHGIGYLPQIPMLEEEEAELEDYSAHCCRWMDCSAVYDHKEELVRHIEKLHVDQRKTEDFTCYWVGCPRNLKPFNARYKLLIHMRVHSGEKPNKCSFEGCKKAFSRLENLKIHLRSHTGEKPYLCQHPGCHKAFSNSSDRAKHQRTHLDTKPYACQVPGCAKRYTDPSSLRKHLKSHSTRERQLRKKMKSYTDGSLDTLTDCLTIQHLQPNTSPLASQDNLSPGASHDSFSAAAQGHDSFGDPHLTHLLPIQDGPRFAAPCPHQVFSEDLCETSPCLTTSGSFLQNNCYLQQFSQAPGVADHSPGFDVHIQEEEITANRELHSAEYYTVLDHGTN

SEQ ID NO: 3 - DNA, 1785 bp, Oryzias latipedes
  CDS (1)..(1785)
atgtttagtggcaaacacgactggccatatggttaccgtgtgcttcctcccctgggatcatcccactgctctaaccaagccaacgagggctgtgagcagttcagtctctccccaggcgctgccatgtcaggcacagcaagcacctccgcctccctcccatcatacctttttgcatatgagactggaagccccagacagacaggtgcgaggaagagacctctctccatgtcacctttgtcggatcttatgggtattgacttcaactctattatacgtacgtcacccacttcccttgtggcttatattaatggtccccgcagttcaccagcctcctactccaccgtctcacccattcagtccgatggatacggtcacttcctgggagtgagaggtcgctgcatacctcagaaccacccctacagttttcccggttcctcccaaattccagccacacagttcgaatgtggccggatggagatgatcgaagagggaagtagtctggaaagccagatggccaacatggtggtgaagcaacagtgcttccctgaggaaattgggtttctggaaaagactacagacagcggcagccaacctagcaacaatgttcttctgtctctacagccagagccagctgcattatccacagtccaggaagatgctgcttcacttgggcctccaccaccctaccactcccacaaacacgtctatctttccagacatcattgcaaagctaaacctccctctaaagaccccgtcacacaacccactgcatatcctcacaagcatggaatcggttatttaccccagattccaatgctggaagaggaagaagcagagttggaggactacagtgctcactgctgccgatggatggactgcagtgcagtttatgaccataaggaggagctggtgaggcacatagagaagctacatgtggaccaaaggaagacggaggacttcacctgctactgggtcggctgtccacgcaacttgaagccctttaatgcccgatacaagcttctcatccacatgagggtccattctggagagaagcccaacaaatgctcgtttgaaggctgcaagaaggcattttctcgacttgaaaacctgaagatccacctgcgcagtcacacgggggagaaaccctatctgtgtcagcacccaggatgccacaaggctttcagcaactccagtgacagagccaaacaccagcgcacacacctggacacaaagccgtacgcatgtcaggttcctggctgtgcaaagcgttacactgatcccagttcattaaggaaacatttgaaatcccactctacaagagagcgacagttacgcaagaagatgaaatcctacactgatggaagtctggacacacttacagattgtttaactatccaacaccttcagccaaacacttcccctctggcaagccaagacaacttatctcctggtgcctctcatgactcattctctgctgcagcacaaggacacgactccttcggcgatcctcacctgactcatttgttacccattcaagatggccccaggtttgctgcaccttgccctcatcaagtgttctctgaggacttgtgtgagacttccccctgcctaaccaccagtggatcattccttcagaacaactgttatctacagcagttcagccaagctcctggtgtggcagatcacagcccaggatttgacgtccacattcaagaggaagaaattactgctaatcgggagctgcacagcgcagagtactacactgttttggaccatggcacaaat

SEQ ID NO: 4 - protein, 595 aa, Oryzias latipedes
MFSGKHDWPYGYRVLPPLGSSHCSNQANEGCEQFSLSPGAAMSGTASTSASLPSYLFAYETGSPRQTGARKRPLSMSPLSDLMGIDFNSIIRTSPTSLVAYINGPRSSPASYSTVSPIQSDGYGHFLGVRGRCIPQNHPYSFPGSSQIPATQFECGRMEMIEEGSSLESQMANMVVKQQCFPEEIGFLEKTTDSGSQPSNNVLLSLQPEPAALSTVQEDAASLGPPPPYHSHKHVYLSRHHCKAKPPSKDPVTQPTAYPHKHGIGYLPQIPMLEEEEAELEDYSAHCCRWMDCSAVYDHKEELVRHIEKLHVDQRKTEDFTCYWVGCPRNLKPFNARYKLLIHMRVHSGEKPNKCSFEGCKKAFSRLENLKIHLRSHTGEKPYLCQHPGCHKAFSNSSDRAKHQRTHLDTKPYACQVPGCAKRYTDPSSLRKHLKSHSTRERQLRKKMKSYTDGSLDTLTDCLTIQHLQPNTSPLASQDNLSPGASHDSFSAAAQGHDSFGDPHLTHLLPIQDGPRFAAPCPHQVFSEDLCETSPCLTTSGSFLQNNCYLQQFSQAPGVADHSPGFDVHIQEEEITANRELHSAEYYTVLDHGTN

SEQ ID NO: 5 - DNA, 2328 bp, Homo sapiens
  CDS (1)..(2328)
atgatggttcagcgactgggactcatttcacctccagcaagccaggtctctacagcatgcaaccagatcagtcctagcttacagagggcaatgaatgcagccaacctgaatatacctccttcagataccaggtcccttatttcgcgtgagtctttggcgtccacgaccttgagtctgacggaaagtcagtcggcctcaagcatgaagcaggagtggtcccagggctacagggccctcccttcgctctccaaccacggctctcagaatggccttgatctaggggatctccttagccttcctcccgggacatccatgtccagcaatagtgtctctaactcattaccatcctacctttttggcacggaaagtagccactctccttaccctagtcctcggcactcatccaccaggtcccactcggcccgctccaagaagagagcgctgtccttgtccccgctgtccgatggcatcgggatagatttcaataccatcatccgcacgtcgcccacgtccttggtggcctacatcaacgggtcgagggcttcgccggccaacctgtccccgcagccggaggtctacgggcatttcctgggcgtgcgcggcagctgcattccccagccgcgcccggtgcccggcagccagaagggcgtgctggtggcccctggaggcctggcgctgccggcctacggcgaggacggggccctggagcacgagcgcatgcaacagctggagcacggcggcctgcagccaggcctggtcaaccacatggtggtgcagcatggcctgccgggccccgacagccagccggccggcctgttcaagaccgaacgcctggaggagttcccgggcagcaccgtagacctaccccccgcgcctccgctccctcctctgccgccgccccaaggccccccacccccttaccatgcccatgcgcaccttcaccacccggagctcgggccccacgcccagcagctggccttgccccaggccaccctggacgacgacggggagatggacggcatcgggggcaagcattgctaccgctggatcgactgcagcgccctgtacgaccagcaggaggagctcgtgcggcacatcgagaaggtccacatcgaccagcgcaaaggggaggacttcacttgcttctgggccggttgccctcgaagatacaagcccttcaacgcccgctataaactgctgatccacatgagagtccactctggggagaagcccaacaagtgtacgtttgaaggttgcgagaaggccttttcaaggcttgaaaatctcaagatccacttgcggagccacacaggcgagaagccgtatttgtgccagcatccgggttgtcagaaggccttcagtaactccagtgaccgcgccaaacaccagcggacgcatctggacaccaaaccttatgcttgtcaaattccaggatgtaccaaacgctacacagacccaagttccctaagaaagcatgtgaaggcacattcttccaaagagcaacaagcaaggaaaaagttgcggtccagcacagagctccatccagacctgctcacagattgcctcaccgtgcagtccctgcagccggccacttcccctagagatgctgctgctgaagggaccgtgggacgctcccctggacccgggcctgacctctattcagctcccattttctccagcaattattcaagccgaagtggaacagctgctggggccgtaccacccccacatcctgtcagtcacccttctccaggacataatgtacaggggagccctcacaacccctcctcccagttacctccactcacagctgtggacgcaggagctgagaggtttgcaccttctgctccatctcctcaccacatcagcccccggagagttccagctccttcttcaatactgcaaagaacacagcctccctatacccagcagccatcaggttcacacctgaagtcctatcagccagaaacaaactcttcttttcaaccaaatggtatccatgtccatggattttatgggcagctgcagaagttctgtcccccacactaccccgattcccagagaattgtgccgcctgtcagctcctgcagtgtggtgccttcgtttgaggactgcctagtccctacatccatgggccaggccagttttgatgttttccacagagccttctcgactcactcgggcattacagtgtatgatttaccttcaagttcctcgagcctctttggggagtctctccgcagcggggctgaagatgctaccttcttgcagatcagcaccgtggaccgctgtcctagccagctctcctctgtctacaccgaaggctaa

SEQ ID NO: 6 - protein, 775 aa, Homo sapiens
MMVQRLGLISPPASQVSTACNQISPSLQRAMNAANLNIPPSDTRSLISRESLASTTLSLTESQSASSMKQEWSQGYRALPSLSNHGSQNGLDLGDLLSLPPGTSMSSNSVSNSLPSYLFGTESSHSPYPSPRHSSTRSHSARSKKRALSLSPLSDGIGIDFNTIIRTSPTSLVAYINGSRASPANLSPQPEVYGHFLGVRGSCIPQPRPVPGSQKGVLVAPGGLALPAYGEDGALEHERMQQLEHGGLQPGLVNHMVVQHGLPGPDSQPAGLFKTERLEEFPGSTVDLPPAPPLPPLPPPQGPPPPYHAHAHLHHPELGPHAQQLALPQATLDDDGEMDGIGGKHCYRWIDCSALYDQQEELVRHIEKVHIDQRKGEDFTCFWAGCPRRYKPFNARYKLLIHMRVHSGEKPNKCTFEGCEKAFSRLENLKIHLRSHTGEKPYLCQHPGCQKAFSNSSDRAKHQRTHLDTKPYACQIPGCTKRYTDPSSLRKHVKAHSSKEQQARKKLRSSTELHPDLLTDCLTVQSLQPATSPRDAAAEGTVGRSPGPGPDLYSAPIFSSNYSSRSGTAAGAVPPPHPVSHPSPGHNVQGSPHNPSSQLPPLTAVDAGAERFAPSAPSPHHISPRRVPAPSSILQRTQPPYTQQPSGSHLKSYQPETNSSFQPNGIHVHGFYGQLQKFCPPHYPDSQRIVPPVSSCSVVPSFEDCLVPTSMGQASFDVFHRAFSTHSGITVYDLPSSSSSLFGESLRSGAEDATFLQISTVDRCPSQLSSVYTEG

SEQ ID NO: 7 - DNA, 2340 bp, Mus musculus
  CDS (1)..(2340)
atggttcagcgactgggacccatttcacctccagcaagccaggtctccacagcatgcaagcagatcagtcctagcttaccgagggcagtgaatgcagccaacctgaatagacctccttcagataccaggtccgttattttgcaagagtctctggtgtccacgactttgagtctgacagagagtcaatcagccttgagtgtgaagcaagagtggtctcagagctacagggctttcccttcactttcatccagccacagttcccagaatggcacggacctaggggaccttcttagcttgcctccaggcacgccagtgtctggcaacagcgtctccaactcgttgccaccctaccttttcggcatggaaaatagccactctccttaccctagccctcggcactcagcaaccagggcccactccacccgctctaagaagagagcattgtccttgtcgccactgtcagatggcatcgggatcgacttcaacactatcatccgtacttcacccacatccttggtcgcctacatcaacggaccaagagcctccccagccaacctgtccccacagtcagaggtctatgggcatttcctgggtgttcgtggcagctgcatcccccagtcttgtgcagtggccagcgggcagaaaggcatattggttgccagtggagggcatacgctgccgggctatggagaggacggtacactggagtatgaacgcatgcagcagcttgagcatggtggcctgcaacccggacctgtaaacaacatggtgttgcagcctggcctaccgggccaggatggccagacagccaacatgctcaaaacagagcgcctggaggagttccccgccagtgccttggacctgccctctgctctgcctctccctcttcctccgcctcagggtcccccacccccataccatgcccatccacaccttcatcacccagagctcctgcctcacacccaatcgctgtccctggcccagactggcctggaagaggatggggagatggaagactcaggggggaagcactgctgccgttggatagactgcagtgccctttatgaccagcaggaggaactggtgaggcacatcgagaaggtccacatagaccaacgcaagggggaagacttcacgtgcttctggactggctgccctagaagatacaagcctttcaacgcacggtataaactgctgatccacatgagggtccactctggggagaagcccaacaagtgtacgttcgaaggctgcaagaaggccttttccaggctcgagaacctcaagattcacttgcggagccacacgggtgagaagccatacttgtgccagcatccgggctgccaaaaggccttcagcaattccagtgaccgtgccaagcaccagaggacacatcttgacactaaaccttatgcttgtcaaattccaggatgtacaaagcgctacacagacccaagctccctcagaaagcacgtgaaggcacattcttcccgagagcagcaagcaaggaaaaagctacggtctagcactgagctccatccagatctcctcacagactgcctcgctgtgcagcccctgcagccagccacgtcccctggagatgctgctgatcataccgtggggcactccccagggccggggcctgggccagggcctggggctgaactctattcagctcccattttcgccagcaatcattcaactcgaagtggaacagctgctggggctgggccacccccacatcctgtcagtcacccttctccaggacataatgtacaggggagcccccacaacccctcctcccagttacctccactcacagctgtggacgcaggcgctgagaggtttgcacctccgactccatctcctcaccacatcagcccgggaagagttccagctcctccgtcactgctccagagagcacaggctccccactcccagcagccaccaggctcactcctgaagccctaccagccagaaaccaactcttcttttcagccaaatggtatccatgtccacggattttatggccagctgcagacattctgtcccccgcattatcctgattcccaaagaactgtgccacccagcggctcctgcagtatggtgccttcctttgaggactgcctggtccccacgtccatgggtcaagccggttttgatgtttttcacagagccttctctacacactcaggcatcacagtgtacgatttgccttcagcttcctcaagcctctttggggaatctcttcgcagtggccctgaagaccccacgttcttgcagctcagtgctgtggaccgctgtcctagccagctctcctctgtgtataccgaaggctga

SEQ ID NO: 8 - protein, 779 aa, Mus musculus
MVQRLGPISPPASQVSTACKQISPSLPRAVNAANLNRPPSDTRSVILQESLVSTTLSLTESQSALSVKQEWSQSYRAFPSLSSSHSSQNGTDLGDLLSLPPGTPVSGNSVSNSLPPYLFGMENSHSPYPSPRHSATRAHSTRSKKRALSLSPLSDGIGIDFNTIIRTSPTSLVAYINGPRASPANLSPQSEVYGHFLGVRGSCIPQSCAVASGQKGILVASGGHTLPGYGEDGTLEYERMQQLEHGGLQPGPVNNMVLQPGLPGQDGQTANMLKTERLEEFPASALDLPSALPLPLPPPQGPPPPYHAHPHLHHPELLPHTQSLSLAQTGLEEDGEMEDSGGKHCCRWIDCSALYDQQEELVRHIEKVHIDQRKGEDFTCFWTGCPRRYKPFNARYKLLIHMRVHSGEKPNKCTFEGCKKAFSRLENLKIHLRSHTGEKPYLCQHPGCQKAFSNSSDRAKHQRTHLDTKPYACQIPGCTKRYTDPSSLRKHVKAHSSREQQARKKLRSSTELHPDLLTDCLAVQPLQPATSPGDAADHTVGHSPGPGPGPGPGAELYSAPIFASNHSTRSGTAAGAGPPPHPVSHPSPGHNVQGSPHNPSSQLPPLTAVDAGAERFAPPTPSPHHISPGRVPAPPSLLQRAQAPHSQQPPGSLLKPYQPETNSSFQPNGIHVHGFYGQLQTFCPPHYPDSQRTVPPSGSCSMVPSFEDCLVPTSMGQAGFDVFHRAFSTHSGITVYDLPSASSSLFGESLRSGPEDPTFLQLSAVDRCPSQLSSVYTEG

SEQ ID NO: 9 - DNA, 21 bp, Artificial Sequence
  synthetic construct
cagcatcttcctggactgtgg

SEQ ID NO: 10 - DNA, 22 bp, Artificial Sequence
  synthetic construct
cacattcgaactgtgtggctgg

SEQ ID NO: 11 - DNA, 23 bp, Artificial Sequence
  synthetic construct
tttgaaggctgcaagaaggcatt

SEQ ID NO: 12 - DNA, 22 bp, Artificial Sequence
  synthetic construct
ctcgacttgaaaacctgaagat

SEQ ID NO: 13 - DNA, 26 bp, Artificial Sequence
  synthetic construct
tcagctccctaaaaacattccaagta

SEQ ID NO: 14 - DNA, 26 bp, Artificial Sequence
  synthetic construct
catcacatataacagcttgcagactg

SEQ ID NO: 15 - DNA, 25 bp, Artificial Sequence
  synthetic construct
ttccctggcctctttagtcagtgac

SEQ ID NO: 16 - DNA, 25 bp, Artificial Sequence
  synthetic primer
aacagtaggtataaattgtgagagg

SEQ ID NO: 17 - DNA, 26 bp, Artificial Sequence
  synthetic construct
cttggagttgttaggggctgtaagag

SEQ ID NO: 18 - DNA, 27 bp, Artificial Sequence
  synthetic construct
cccaccattgcattgaagtgttctccc

SEQ ID NO: 19 - DNA, 30 bp, Artificial Sequence
  synthetic construct
ggtcaataaataaatagttaccagtataaa

SEQ ID NO: 20 - DNA, 28 bp, Artificial Sequence
  synthetic construct
ctgaaatctgccacaatcatgtaactgc

SEQ ID NO: 21 - DNA, 26 bp, Artificial Sequence
  synthetic construct
tgtggtaatggtttcatggtctgggc

SEQ ID NO: 22 - DNA, 26 bp, Artificial Sequence
  synthetic construct
gcagtagttctttagaaattccgatc

SEQ ID NO: 23 - DNA, 23 bp, Artificial Sequence
  synthetic construct
cactgctgccgatggatggactg

SEQ ID NO: 24 - DNA, 23 bp, Artificial Sequence
  synthetic construct
ttgttgggcttctctccagaatg

SEQ ID NO: 25 - DNA, 23 bp, Artificial Sequence
  synthetic construct
tttgaaggctgcaagaaggcatt

SEQ ID NO: 26 - DNA, 22 bp, Artificial Sequence
  synthetic construct
cttgcgtaactgtcgctctctt

SEQ ID NO: 27 - DNA, 21 bp, Artificial Sequence
  synthetic construct
cgttgcgcagatatacgtcac

SEQ ID NO: 28 - DNA, 21 bp, Artificial Sequence
  synthetic construct
aggcgtctctccagtagcttg

SEQ ID NO: 29 - DNA, 23 bp, Artificial Sequence
  synthetic construct
cactgctgccgatggatggactg

SEQ ID NO: 30 - DNA, 22 bp, Artificial Sequence
  synthetic construct
cttgcgtaactgtcgctctctt

SEQ ID NO: 31 - DNA, 23 bp, Artificial Sequence
  synthetic construct
tttgaaggctgcaagaaggcatt

SEQ ID NO: 32 - DNA, 24 bp, Artificial Sequence
  synthetic construct
ctttcattattcaagaaacaggtg

SEQ ID NO: 33 - DNA, 23 bp, Artificial Sequence
  synthetic construct
tttgaaggctgcaagaaggcatt

SEQ ID NO: 34 - DNA, 22 bp, Artificial Sequence
  synthetic construct
aacaatctgtaagtgtgtccag

SEQ ID NO: 35 - DNA, 21 bp, Artificial Sequence
  synthetic construct
tgagtgttgcaacaggccatc

SEQ ID NO: 36 - DNA, 22 bp, Artificial Sequence
  synthetic construct
cacattcgaactgtgtggctgg

SEQ ID NO: 37 - DNA, 23 bp, Artificial Sequence
  synthetic construct
cactgctgccgatggatggactg

SEQ ID NO: 38 - DNA, 23 bp, Artificial Sequence
  synthetic construct
ttgttgggcttctctccagaatg

SEQ ID NO: 39 - DNA, 23 bp, Artificial Sequence
  synthetic construct
cactgctgccgatggatggactg

SEQ ID NO: 40 - DNA, 22 bp, Artificial Sequence
  synthetic construct
cttgcgtaactgtcgctctctt

SEQ ID NO: 41 - DNA, 23 bp, Artificial Sequence
  synthetic construct
cactgctgccgatggatggactg

SEQ ID NO: 42 - DNA, 22 bp, Artificial Sequence
  synthetic construct
gccagagctgtctgctgtgacg

SEQ ID NO: 43 - DNA, 20 bp, Artificial Sequence
  synthetic construct
caggacgtctacaaaatcgg

SEQ ID NO: 44 - DNA, 21 bp, Artificial Sequence
  synthetic construct
agctcgttgaacttgcaggcg

SEQ ID NO: 45 - DNA, 23 bp, Artificial Sequence
  synthetic construct
cactgctgccgatggatggactg

SEQ ID NO: 46 - DNA, 22 bp, Artificial Sequence
  synthetic construct
aattcctcagctggacactggc

SEQ ID NO: 47 - DNA, 22 bp, Artificial Sequence
  synthetic construct
ttacctcttctaagagctcagg

SEQ ID NO: 48 - DNA, 22 bp, Artificial Sequence
  synthetic construct
tgcgctggtgtttggctctgtc

SEQ ID NO: 49 - DNA, 22 bp, Artificial Sequence
  synthetic construct
caccaagtggttggtaacggag

SEQ ID NO: 50 - DNA, 23 bp, Artificial Sequence
  synthetic construct
attcttccaaaagcgcattggtg

SEQ ID NO: 51 - DNA, 21 bp, Artificial Sequence
  synthetic construct
cgacctcatcctggtggacga

SEQ ID NO: 52 - DNA, 21 bp, Artificial Sequence
  synthetic construct
ctgagacaccacgtggtaagc

SEQ ID NO: 53 - DNA, 21 bp, Artificial Sequence
  synthetic construct
aaggcttacttgtgtctggac

SEQ ID NO: 54 - DNA, 21 bp, Artificial Sequence
  synthetic construct
tccagctcgtccagtgacatg

SEQ ID NO: 55 - DNA, 18 bp, Artificial Sequence
  synthetic construct
cactcatgtctaaaacgg

SEQ ID NO: 56 - DNA, 18 bp, Artificial Sequence
  synthetic construct
actaaacatggactgtgt